(19) United States
(12) Patent Application Publication
(10) Pub. No.: US 2026/0192154 A1
(43) Pub. Date: Jul. 9, 2026

(54) METHODS AND SYSTEMS OF FACILITATING PERFORMANCE EVALUATION OF PHYSICAL ACTIVITIES

(71) Applicant: Neotericc LLC, LONE TREE, CO (US)

(72) Inventor: Patrick L. Carter, Lone Tree, CO (US)

(73) Assignee: Neotericc LLC, LONE TREE, CO (US)

(21) Appl. No.: 19/435,924

(22) Filed: Dec. 30, 2025

Related U.S. Application Data
(60) Provisional application No. 63/739,812, filed on Dec. 30, 2024.

(51) Int. Cl. A63B 24/00; G16H 40/67; H04L 67/12

(52) U.S. Cl. CPC A63B 24/0062; G16H 40/67; H04L 67/12; A63B 2024/0068; A63B 2225/20

(57) ABSTRACT

The present disclosure provides a method of facilitating performance evaluation of physical activities. Further, the method includes receiving, using a communication device, a content data from a content source device. Further, the content data corresponds to a physical activity. Further, the method includes determining, using a processing device, a characteristic data using an Artificial Intelligence (AI) model based on the content data. Further, the characteristic data includes an actual metric of a biomechanical characteristic of the physical activity. Further, the method includes analyzing, using the processing device, the characteristic data and a reference data. Further, the reference data includes a reference metric of the biomechanical characteristic. Further, the method includes generating, using the processing device, a feedback data based on the analyzing. Further, the feedback data includes a feedback on the physical activity. Further, the method includes transmitting, using the communication device, the feedback data to a user device.

### REFERENCE TO RELATED APPLICATIONS

This application claims the benefit of U.S. Provisional Patent Application No. 63/739,812, titled “METHODS AND SYSTEMS OF PROVISIONING A PERFORMANCE EVALUATION OF A PHYSICAL ACTIVITY”, filed on Dec. 30, 2024, which is incorporated by reference herein in its entirety.

### FIELD OF DISCLOSURE

The present disclosure relates to the field of data processing. More specifically, the present disclosure relates to systems and methods of facilitating performance evaluation of physical activities.

### BACKGROUND

The present disclosure relates generally to the field of biomechanical performance evaluation and digital sports analytics, a field that has become increasingly important as athletes, trainers, and coaches rely on data-driven insights to improve technique, reduce injury risk, and enhance overall performance. As sports training environments continue to evolve toward remote coaching, personalized analytics, and real-time feedback systems, there is a growing need for platforms capable of accurately interpreting physical activities using digital content such as video streams or sensor-derived data. The ability to analyze human movement with precision, assess subtle mechanical variations, and provide actionable guidance has become a critical objective in optimizing athletic performance, rehabilitation, and skill development across a wide range of physical disciplines.

A desirable objective in the field of biomechanical performance evaluation and digital sports analytics is to provide a mechanism that may evaluate the performance of a physical activity in a manner that is accurate, personalized, context-aware, responsive, and capable of adapting to an individual's changing biomechanical profile over time. Such an evaluation should ideally capture nuanced aspects of motion dynamics, generate meaningful metrics and scores, and deliver feedback in a timely manner so that athletes may apply corrective actions during training. Additionally, that evaluation should be robust across different sporting contexts, incorporate both human movement and equipment interaction, and function effectively without restrictive hardware setups or controlled environments.

However, existing approaches face several limitations in achieving the above objective. Many known systems rely heavily on coarse or static measurements that fail to capture the multi-stage nature of athletic movements. The existing approaches often struggle to analyze fine-grained biomechanical characteristics or to distinguish between different phases of a complex activity. Further, the existing approaches may require extensive manual observation or specialized hardware, which may restrict usability in real-world settings. Current methods may also lack adaptability over time, offering limited ability to account for variations due to injury recovery, training progression, or changes in technique.

Feedback generated by such systems may be delayed, insufficiently personalized, or unsupported by contextual understanding of the surrounding environment or scenario in which the action occurs. Furthermore, certain solutions may be unable to integrate or interpret the mechanics of associated sports equipment in relation to an athlete's motion, thereby providing an incomplete assessment. Some techniques may also be limited in the capability to handle uncertainty, to perform scalable computation, or to operate efficiently in environments with bandwidth or latency constraints.

Further, existing performance evaluation systems primarily focus on high-level metrics like success rates (e.g., shooting percentage) or rely heavily on manual observation. Further, the approach of focusing on the high-level metric often fail to notice the importance of detailed biomechanical analysis across multiple phases of motion. Furthermore, static baselines of the existing performance evaluation systems may not account for an athlete's progress over time, including recovery from injury or evolving coaching strategies.

Therefore, there is a need for improved systems and methods of facilitating performance evaluation of physical activities that may overcome one or more of the preceding problems.

### SUMMARY OF DISCLOSURE

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

The present disclosure provides a method of facilitating performance evaluation of physical activities. Further, the method may include receiving, using a communication device, one or more content data from one or more content source devices. Further, the one or more content data corresponds to one or more physical activities. Further, the method may include determining, using a processing device, one or more characteristic data using one or more Artificial Intelligence (AI) models based on the one or more content data. Further, the one or more characteristic data includes one or more actual metrics of one or more biomechanical characteristics of the one or more physical activities. Further, the method may include analyzing, using the processing device, the one or more characteristic data and one or more reference data. Further, the one or more reference data includes one or more reference metrics of the one or more biomechanical characteristics of the one or more physical activities. Further, the method may include generating, using the processing device, a feedback data based on the analyzing of the one or more characteristic data. Further, the feedback data includes a feedback on the one or more physical activities. Further, the method may include transmitting, using the communication device, the feedback data to one or more user devices.

The present disclosure provides a system for facilitating performance evaluation of physical activities. Further, the system may include a communication device. Further, the communication device may be configured for receiving one or more content data from one or more content source devices. Further, the one or more content data corresponds to one or more physical activities. Further, the communication device may be configured for transmitting a feedback data to one or more user devices. Further, the system may include a processing device communicatively coupled with the communication device. Further, the processing device may be configured for determining one or more characteristic data using one or more Artificial Intelligence (AI) models based on the one or more content data. Further, the one or more characteristic data includes one or more actual metrics of one or more biomechanical characteristics of the one or more physical activities. Further, the processing device may be configured for analyzing the one or more characteristic data and one or more reference data. Further, the one or more reference data includes one or more reference metrics of the one or more biomechanical characteristics of the one or more physical activities. Further, the processing device may be configured for generating the feedback data based on the analyzing of the one or more characteristic data. Further, the feedback data includes a feedback on the one or more physical activities.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTIONS OF DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

The drawings presented with this disclosure may illustrate representative and non-limiting arrangements of hardware components, software modules, artificial intelligence subsystems, machine learning architectures, data processing pipelines, user interfaces, network topologies, and memory arrangements that may be used to understand embodiments of the present subject matter. They may depict functional or conceptual layouts intended to facilitate explanation of the disclosed principles. The geometric appearance, dimensional proportions, ordering, grouping, and naming of elements within the drawings are not intended to imply any restriction on implementation. The drawings may schematically portray computing environments containing client devices, servers, distributed computing clusters, communication networks, storage systems, or artificial intelligence models arranged for training, inference, or combined operations. The drawings may include simplified symbolic representations of algorithmic processes, workflows, blocks, or modules; such symbolic representations are treated as abstractions of underlying hardware and software operations rather than literal structural requirements. Similarly, lines connecting components may represent logical associations, communication pathways, or data relationships rather than any specific physical wiring or layout. These figures may also illustrate non-exhaustive examples of operational stages, sequencing, or interactions among artificial intelligence components such as encoders, decoders, generators, discriminators, featurizers, transformers, or safety-validation modules. Any specific combination or configuration shown is presented for explanatory clarity only. Additional drawings, alternative views, or more granular depictions may be used without affecting the scope of the claims.

FIG. 1 is an illustration of an online platform 100 consistent with various embodiments of the present disclosure.

FIG. 2 is a block diagram of a computing device 200 for implementing the methods disclosed herein, in accordance with some embodiments.

FIG. 3 is a block diagram illustrating a machine-learning system 300 for implementing various embodiments of this disclosure, in accordance with some embodiments.

FIG. 4 illustrates a flowchart of a method 400 of facilitating performance evaluation of physical activities, in accordance with some embodiments.

FIG. 5 illustrates a flowchart of a method 500 of facilitating performance evaluation of physical activities including generating, using the processing device 804, the at least one reference data, in accordance with some embodiments.

FIG. 6 illustrates a flowchart of a method 600 of facilitating performance evaluation of physical activities including identifying, using the processing device 804, the at least one reference data from the plurality of reference data, in accordance with some embodiments.

FIG. 7 illustrates a flowchart of a method 700 of facilitating performance evaluation of physical activities including generating, using the processing device 804, the at least one reference data, in accordance with some embodiments.

FIG. 8 illustrates a block diagram of a system 800 of facilitating performance evaluation of physical activities, in accordance with some embodiments.

FIG. 9 illustrates a block diagram of the system 800 of facilitating performance evaluation of physical activities, in accordance with some embodiments.

FIG. 10 illustrates a flowchart 1000 of multi-phase evaluation of physical activities, in accordance with some embodiments.

FIG. 11 illustrates a spatial-temporal mechanics of a player 1100 playing Hockey, in accordance with some embodiments.

FIG. 12 illustrates a graph 1200 of quaternion rotation dynamics, in accordance with some embodiments.

FIG. 13 illustrates a Gaussian distribution graph 1302 of physical activities, in accordance with some embodiments.

FIG. 14 illustrates a flowchart of a method 1400 of provisioning a performance evaluation of a physical activity, in accordance with some embodiments.

FIG. 15 illustrates a flowchart of a method 1500 of provisioning a performance evaluation of a physical activity including generating, using the processing device 804, at least one metric associated with the plurality of characteristics, in accordance with some embodiments.

FIG. 16 illustrates a flowchart of a method 1600 of provisioning a performance evaluation of a physical activity including generating, using the processing device 804, at least one target metric corresponding to the physical activity, in accordance with some embodiments.

FIG. 17 illustrates a flowchart of a method 1700 of provisioning a performance evaluation of a physical activity including generating, using the processing device 804, a score data, in accordance with some embodiments.

FIG. 18 illustrates a flowchart of a method 1800 of facilitating performance evaluation of physical activities, in accordance with some embodiments.

### DETAILED DESCRIPTION OF DISCLOSURE

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of the disclosed use cases, embodiments of the present disclosure are not limited to use only in this context.

The detailed description that follows may provide a framework for describing computer-implemented systems, artificial intelligence systems, distributed learning infrastructures, data processing pipelines, and hardware and software arrangements suitable for implementing embodiments shown in the drawings. Terms such as processing, computing, determining, or generating refer to actions performed by computing systems or electronic devices that manipulate data represented as physical signals, stored values, or encoded information within registers, memory structures, and storage devices.

The present disclosure contemplates implementations involving artificial intelligence, machine learning, distributed computation, and computer-implemented systems operating upon data represented as physical electronic or optical signals. Descriptions of processing, analyzing, determining, transforming, encoding, decoding, generating, inferring, synthesizing, modifying, storing, retrieving, ranking, filtering, validating, classifying, or otherwise manipulating information are to be understood as referring to the actions of computing systems, electronic devices, or computational circuits that manipulate such signals in memory elements, registers, buffers, or storage media. These operations may be performed by general-purpose processors, specialized processors, machine learning accelerators, or combinations thereof.

The disclosure contemplates implementations in which artificial intelligence systems perform perception, synthesis, inference, prediction, or generation of information using models whose configurations may evolve based on training, feedback, or adaptive learning processes. A model may initially be configured with a set of parameters and architectural structures that define its behavior, and this configuration may change automatically as the model encounters training inputs, validation data, reference data, or instructor-provided feedback. A machine learning model may modify its internal state through optimization techniques, gradient updates, reinforcement signals, vector transformations, attention mechanisms, latent variable adjustments, embedding refinements, or other learning operations executed electronically. Such modifications may occur over extended cycles, partial cycles, or continual learning sequences without explicit intervention by a human.

The disclosure contemplates systems involving data ingestion pipelines that gather input from sources including but not limited to sensor signals, event streams, text data, image data, audio data, video data, structured and unstructured repositories, application logs, telemetric feeds, network services, or human-generated content. Ingestion functions may include filtering, normalization, augmentation, segmentation, batching, tokenization, windowing, compression, encryption, decryption, hashing, deduplication, contextualization, and mapping to internal formats. Intermediate components may transform this data into derived representations, including embeddings, latent encodings, feature tensors, multi-modal joint representations, or contextual vectors suitable for use by downstream modeling engines. These transformations may be performed using neural networks, statistical encoders, dimensionality-reduction algorithms, or hybrid computational modules.

The disclosure contemplates machine learning systems that may employ advanced architectures such as transformer networks, encoder-decoder stacks, mixture-of-experts structures, diffusion models, recurrent networks, convolutional hierarchies, attention-based models, retrieval-augmented architectures, cross-modal alignment engines, graph neural networks, probabilistic models, auto-encoding frameworks, or hybrid symbolic-neural systems. Such models may implement deep layers configured to perform operations including attention calculations, feed-forward projections, gating operations, positional encoding, normalization steps, multi-head routing, sequential decoding, or latent pathway selection. Multi-modal systems may combine textual, visual, auditory, sensory, or structured inputs within joint representational spaces. Embeddings may be learned from large corpora or multi-modal datasets and may encode semantic, syntactic, structural, temporal, spatial, or contextual relationships across modalities. These embeddings may be dynamically updated as the system encounters new information, thereby improving consistency, expressiveness, or alignment with real-world contexts.

The disclosure contemplates training processes that may involve supervised learning, unsupervised learning, semi-supervised learning, self-supervised learning, reinforcement learning, preference optimization, curriculum-based learning, active learning, or continual learning. Training operations may include forward passes through the model, backward propagation of gradients, update steps using optimization algorithms, adaptive learning-rate scheduling, regularization steps, loss-function evaluation, and checkpointing of intermediate states. Training datasets may include real-world data, synthetic data, simulated data, augmented data, or mixtures thereof. Validation procedures may evaluate performance metrics, generalization behavior, safety constraints, or compliance with domain-specific criteria. In some implementations, refinement cycles may incorporate human-in-the-loop interventions, reward model shaping, safety evaluator feedback, or guided corrections.

The disclosure contemplates distributed or federated execution in which computation is partitioned across multiple hardware devices, regions, or clusters. Certain operations may occur at edge devices for low latency, while others may be delegated to remote servers, cloud clusters, datacenters, or specialized compute fabrics. Components may communicate over wired or wireless networks supporting data exchange, synchronization, replication, or model-state updates. Distributed learning processes may synchronize gradients, coordinate model versions, merge updates across shards, or exchange activation values within parallel training regimes. Distributed inference may involve routing requests across replicas, balancing load through orchestration layers, or selecting model pathways dynamically. Network connections may include encryption, authentication, secure session management, or routing protocols appropriate for maintaining privacy, integrity, or availability.

The disclosure contemplates orchestration layers capable of managing complex workflows involving model invocation, tool invocation, external data retrieval, decision routing, fallback selection, multi-model aggregation, post-processing evaluation, or safety governance. Orchestration environments may evaluate contextual signals, metadata, user characteristics, or policy constraints to determine which models, subsystems, or computational branches should be executed. Such environments may dynamically alter execution pathways based on estimated performance, resource availability, model confidence, safety risk, or real-time system health. Post-processing components may evaluate generated outputs for compliance with content policies, statutory requirements, operational constraints, or domain-specific decision rules.

The disclosure contemplates safety-oriented components that evaluate model outputs or intermediate representations for consistency with safety criteria, quality thresholds, regulatory considerations, factual accuracy constraints, domain restrictions, or alignment requirements. Safety modules may employ auxiliary models, discriminators, rule sets, statistical detectors, confidence estimators, or hybrid evaluators to identify undesirable outputs. These modules may trigger remediation actions including output modification, output rejection, re-routing through alternate inference pathways, invocation of corrective models, or escalation for human review. Safety processes may incorporate real-time validation, contextual scoring, adversarial robustness analysis, anomaly detection, or controlled generation constraints.

The disclosure contemplates governance structures including policy managers, audit loggers, compliance trackers, version controllers, and provenance systems that associate model outputs with contextual metadata, historical signals, update events, training sources, or safety evaluations. Systems may maintain lineage records documenting which model version, configuration state, or training dataset contributed to an outcome. Governance modules may ensure that system behavior aligns with formal requirements such as fairness principles, legal obligations, industry standards, or institutional guidelines.

The disclosure contemplates storage and memory systems capable of storing model parameters, datasets, embeddings, logs, metrics, checkpoints, execution traces, and auxiliary information used to configure or interpret model behavior. These storage systems may include magnetic media, semiconductor memory, optical media, solid-state arrays, distributed storage fabrics, or hybrid memory hierarchies. Storage media may contain instructions, configurations, or data structures that, when accessed by a computing device, configure that device to carry out the operations described herein. Such media may include executables, bytecode, machine code, firmware, microcode, program modules, configuration files, architectural descriptors, or schema definitions.

The disclosure contemplates user interfaces that permit human operators to view model outputs, initiate tasks, modify configurations, inspect metrics, interact with logs, evaluate safety signals, or guide system adaptation. Interfaces may be multimodal and may support textual input, speech commands, visual interaction, gesture control, or programmatic invocation through APIs. Administrative interfaces may allow for reviewing system performance, tuning operational thresholds, enabling or disabling features, monitoring resource use, examining generated content, or initiating refinement workflows.

The disclosure contemplates systems in which instructions are executed entirely on a single device, partially on multiple devices, or cooperatively across remote and local environments. Code may execute directly on hardware, within firmware, inside virtual machines, inside containers, or through any combination of software and hardware interactions. Computational instructions may be stored locally, transferred via communication networks, or streamed from remote systems. Implementations may involve software executing on general-purpose processors, specialized logic circuits performing equivalent functions, or hybrid mechanisms that combine hardware acceleration with software guidance.

Interpretation of terms in this disclosure is governed by principles commonly applied by persons of ordinary skill in the relevant field. Technical and scientific terms used herein should be understood in a manner consistent with their usage in the field of artificial intelligence, machine learning, computing, networking, data storage, or any related discipline. Terms describing functionality should not be interpreted as strictly structural unless explicitly stated. Phrases such as configured to, adapted to, operable to, or capable of indicate permissible functionality rather than structural limitations. Terms such as a or an encompass one or more unless clearly contradicted by context. Terms joined by or should be interpreted as inclusive, and terms joined by and should be interpreted as collective.

The description set forth herein provides a broad and flexible framework intended to support a wide range of computer-implemented, machine-learning-enabled, distributed, and multimodal embodiments. Variations may include reallocation of tasks, substitution of algorithms, reconfiguration of models, changes to pipeline ordering, or adoption of alternate hardware. No combination or arrangement mentioned herein should be regarded as required unless explicitly stated. The scope of protection is established by the claims, interpreted in light of this description.

The present disclosure contemplates implementations that employ advanced mathematical frameworks characteristic of modern artificial intelligence systems. Machine learning models may be conceptualized as parameterized functions that map elements of an input space to elements of an output space. Such a function may be defined over real-valued, complex-valued, vector-valued, tensor-valued, or mixed-modal domains. The model may implement successive transformations applied to an ordered set of input vectors using compositions of linear operators, nonlinear activations, attention functions, normalization operations, and dimensional projections.

Model parameters may be represented as ordered collections of real-valued scalars arranged into structures such as matrices, tensors, kernels, filters, or embeddings. These parameters may be optimized by minimizing a loss functional defined over an expected distribution of input-output pairs. The optimization process may involve computing gradients of the loss functional with respect to each model parameter, followed by an update step that serves to reduce the value of the loss functional. Gradient computation may use automatic differentiation frameworks that symbolically or numerically propagate partial derivatives backward through a computational graph.

Attention mechanisms may employ a similarity measure between projected query vectors and projected key vectors. This similarity measure may yield a weight distribution over contextual elements. The weighted combination of projected value vectors may form an attention output that is subsequently transformed through additional layers. Multiple independent attention heads may be aggregated to capture heterogeneous relationships within the input domain. Cross-attention mechanisms may operate similarly but with distinct source and target sequences.

Normalization steps may rescale intermediate representations using learned scaling and shifting coefficients. Activation functions may introduce nonlinearity by applying element-wise transformations selected to ensure differentiability and expressive capacity. Residual pathways may combine transformed and untransformed representations to facilitate stable gradient propagation under deep compositions. Positional encodings or structural embeddings may inject ordering, spatial, temporal, or relational information into otherwise permutation-invariant architectures.

Multi-modal models may operate over domains that combine text, image, audio, video, sensor, or structured signals. These domains may be embedded into a common vector space through learned projection operators. Joint training processes may enforce alignment constraints that minimize representational divergence between modalities while preserving intra-modal semantics.

Diffusion frameworks may model data generation as the reversal of a stochastic corruption process. A forward process may incrementally add noise to data samples, while a learned reverse process may approximate the time-reversed conditional probability distribution. The reverse process may be parameterized by a neural network trained to denoise intermediate states. Continuous-time formulations may model this process using stochastic differential equations whose drift and diffusion terms are learned through score-matching or related techniques.

Reinforcement-based procedures may model learning as an optimization of expected reward under a policy function. The policy may produce distributions over actions given a latent or explicit representation of the environment state. Policy gradients may be estimated from sampled trajectories, and advantage estimators may reduce variance of such gradients. Value functions may approximate the expected cumulative reward, and these approximations may be updated through temporal-difference learning.

Generative models may be expressed in probabilistic terms as joint or conditional distributions parameterized by neural architectures. Such models may perform sampling by iteratively drawing latent variables from a learned distribution and transforming those variables into output space. Variational models may introduce auxiliary latent variables whose posterior distributions are approximated through recognition functions that optimize an evidence-bound objective.

Matrix decompositions, spectral analysis, manifold learning, kernel operators, and other mathematical constructs may be incorporated to improve expressiveness, stability, or computational efficiency. Training may involve sophisticated schedulers, trust-region constraints, adaptive learning-rate schemes, gradient-norm clipping, regularization penalties, entropy maximization, attention masking, or mixed-precision arithmetic.

All such mathematical constructs are conceptual, descriptive, and non-limiting. The disclosure encompasses any differentiable or non-differentiable optimization method, any discrete or continuous learning paradigm, and any representational transformation that may be understood by a person of ordinary skill in the field.

Further, the disclosure provides a computing environment which may include a combination of client devices, servers, distributed computing clusters, databases, external data sources, network nodes, and interface endpoints. Such an environment may support artificial intelligence workloads including perception, synthesis, inference, prediction, and generation, using hardware and software foundations designed for high-throughput and low-latency operation. Embodiments may involve the coordinated use of multiple machine learning models, whose configurations may evolve over time as they learn from training, validation, reference, or feedback data. Models may adjust their internal parameters through supervised, unsupervised, or reinforcement-based processes, allowing automatic electronic improvements to their performance based on input data and observed outcomes.

Further, the disclosure provides a computing device which may include processing units, memory elements, storage devices, system buses, high-speed controllers, low-speed controllers, and expansion interfaces. Processors may include general-purpose units, multi-core processors, vector processors, digital signal processors, tensor accelerators, neural accelerators, graphics engines, or various kinds of specialized integrated circuits including FPGAs, ASICs, ASSPs, SoCs, and CPLDs. A device may include system memory composed of volatile or non-volatile components such as RAM, DRAM, flash memory, ROM, or phase-change memory. The storage subsystem may include solid-state drives, magnetic disks, optical media, arrays of storage devices, and network-attached storage resources. Input and output mechanisms may include microphones, displays, keyboards, pointing devices, biometric sensors, gesture or touch interfaces, and actuators suitable for multimodal interaction with a user.

Further, the disclosure provides a machine-learning architecture which may include engines or modules such as a data input engine, data retrieval engine, data transform engine, featurization engine, modeling engine, generative engine, validation engine, feedback engine, and refinement engine. A data input pipeline may obtain structured or unstructured information from various sources, transform the information into model-compatible forms, and store such transformed data in memory or storage accessible to downstream components. A modeling engine may perform tasks such as model training, re-configuration, validation, and testing, executing iterative processes across multiple cycles or passes through training data. A predictive or generative engine may construct outputs based on intermediate representations, learned embeddings, or latent encodings generated by layers such as encoder-decoder structures, attention mechanisms, or multi-layer transformer architectures. Embeddings may represent discrete entities such as words, documents, or images as continuous vectors in high-dimensional spaces, capturing semantic or structural relationships useful for downstream tasks.

Further, the disclosure may provide a distributed or cloud-based operation may include multiple physical or virtual instances of computing devices, distributed across data centers or network boundaries. Functions may be partitioned across machines to achieve parallelism, redundancy, fault tolerance, or improved throughput. Distributed systems may use load balancing mechanisms to maintain stable processing, memory, or bandwidth utilization across clusters and avoid overload conditions. Such deployments may require communication over wired or wireless networks that implement a variety of protocols including HTTP, HTTPS, MQTT, CoAP, or any other suitable communication framework. Communication channels may include local networks, wide-area networks, personal-area networks, or global communication systems, potentially utilizing secure encrypted sessions such as SSL-based channels.

Further, the disclosure may provide an algorithm, process, or flow diagram which may include operations that may occur in sequences, reversed orders, concurrently, or in partially overlapping timelines, depending on the implementation. Blocks representing actions in a flowchart may correspond to program modules, instruction sequences, or hardware logic capable of performing the specified acts. Such operations may manipulate physical quantities such as electrical or magnetic signals stored or transferred among memory units, registers, storage devices, or communication media. Flow diagrams may be realized through software running on general-purpose processors, through dedicated hardware circuits, or through combinations of both.

Further, the disclosure may provide memory, storage, or programmatic constructs which may include program instructions encoded on computer-readable media including electronic, magnetic, optical, electromagnetic, semiconductor, or other tangible media. Examples include RAM, ROM, EEPROM, flash memory, magnetic disks, optical disks, and mechanical encoded structures such as punch cards or raised-pattern media. Such storage media may store instructions that, when executed, configure the memory and therefore configure the computing device itself, causing the device to perform functions described in association with the drawings.

Further, the disclosure may provide a user interface which may include graphical displays, dashboards, selection controls, input fields, monitoring elements, or multimodal interaction surfaces, allowing users to interact with computing systems in speech, touch, gesture, or other modalities. Such interfaces may be presented through client devices, server applications, or remote access platforms and may support visualization of model behavior, systems performance, or configuration parameters.

Further, described features may be combined, rearranged, omitted, or substituted without departing from the principles disclosed. Variations may involve distributing functionality across devices, merging components, implementing features in hardware rather than software, or employing alternative communication protocols. Many such variations and modifications are intended to fall within the scope of the disclosure as understood by persons skilled in the art.

The detailed description of the drawings therefore provides a foundation for describing technical, architectural, and operational aspects of embodiments, while allowing broad flexibility in how such embodiments may be implemented in practice. The scope of such embodiments is governed by the claims rather than the illustrative content of the drawings.

In some embodiments, a system consistent with this disclosure includes one or more client devices, one or more servers, and one or more data stores coupled by one or more networks. The client devices can include, without limitation, mobile phones, tablet computers, laptop or desktop computers, wearable devices, smart displays, vehicles, robots, or other computing platforms equipped with data processing hardware and memory hardware. The servers can include data servers, application servers, web servers, proxy servers, or cloud computing services that provide shared processing, storage, and networking resources. The data stores can include databases, object stores, file systems, or other repositories that persist configuration data, training data, logs, model artifacts, and other information.

The networks can include public and private networks, such as local area networks, wide area networks, and cloud networks, using wired or wireless communication links. The networks can provide routing, addressing, access control, encryption, and related functionality using standard or proprietary protocols.

Each computing device, whether a client device or a server, can include one or more processors, system memory, persistent storage, communication interfaces, and input or output devices. The processors can include general purpose central processing units, graphics processing units, digital signal processors, microcontrollers, application specific integrated circuits, field programmable gate arrays, or other programmable or fixed function processing elements configured to execute instructions or perform logic operations. The memory can include volatile and non-volatile storage, such as random access memory and read only memory. The persistent storage can include solid state drives, magnetic disks, optical media, or other non-transitory computer readable media.

Program code executed by the processors can include operating systems, device drivers, libraries, and application programs, including components that implement portions of the methods described herein. Program code and data can be stored on computer readable media and loaded into memory by standard mechanisms, such as boot loaders, installation programs, or update services.

Input devices can include keyboards, pointing devices, microphones, cameras, touch sensitive surfaces, biometric sensors, and other sensors. Output devices can include displays, speakers, haptic devices, printers, and other actuators. Some devices can support multimodal interaction, allowing combined or sequential input and output through various modalities.

For purposes of this disclosure, artificial intelligence systems may include arrangements of software and hardware that perform tasks such as perception, prediction, planning, or generation based on input data. These systems can employ one or more models, such as statistical models, neural networks, decision trees, or other machine learning models. As used herein, a “model” can refer to a parameterized function, an ensemble of such functions, or a collection of cooperating components that process data and produce outputs.

In some embodiments, the system includes a data input engine that obtains data from one or more sources, such as application logs, sensor streams, structured databases, and unstructured content. The data input engine can retrieve, filter, aggregate, or transform the data into feature representations suitable for model consumption. Data sources can include training data, validation data, and reference data used to evaluate and calibrate model behavior.

A modeling engine can manage one or more training processes for one or more models. The modeling engine can select model architectures, initialize parameters, and apply training algorithms such as supervised learning, semi supervised learning, unsupervised learning, reinforcement learning, or combinations thereof. The modeling engine can also manage hyperparameters, training schedules, and evaluation procedures across epochs or passes through the data.

The system can include a generative response engine or inference engine that receives prompts or other inputs and generates outputs using one or more models. For example, a natural language interface can receive a text prompt, embed or otherwise encode the prompt, process the encoded prompt using a transformer based model or other sequence model, and generate a sequence of tokens that are decoded into an output. The engine can generate multiple candidate outputs and apply validation or ranking logic to select a final result according to quality, safety, or relevance criteria.

A feedback engine can collect explicit or implicit feedback signals, such as user ratings, corrective edits, or outcome metrics derived from downstream tasks. A refinement engine can use such feedback to adjust model parameters, routing logic, or policies, for example by performing additional training steps, updating reward models, or modifying configuration parameters.

In certain embodiments, the disclosed techniques are applied to platforms that include sensors and actuators, such as vehicles, robots, or other machines. The platform can include a processor system that receives signals from cameras, LIDAR units, radar units, inertial sensors, and other devices, and produces control outputs for steering, propulsion, braking, or other actuators. Sensor data can be captured at various sampling rates and processed by perception models to detect and track objects and infer scene attributes.

Planning and control components can receive outputs from perception models along with route information, traffic rules, and high level goals. These components can generate trajectories or control commands, optionally using reinforcement learned policies, optimization based planners, or hybrid systems. Connections to backend services can permit off board processing, fleet level learning, or remote supervision where appropriate, while on board components can maintain safe operation in the presence of network latency or failures.

The systems described herein can be implemented using centralized, decentralized, or hybrid arrangements. For instance, models may be deployed in cloud environments, on edge devices, or across both, depending on requirements such as latency, privacy, cost, and reliability. Load balancing and resource management components can distribute processing across devices or data centers and can provide elasticity to accommodate changing workloads.

Certain embodiments may expose functionality through application programming interfaces, software development kits, or graphical user interfaces. Client applications can submit requests to backend services, which can apply authentication, authorization, logging, and policy enforcement before invoking models or tools and returning results.

The systems and methods disclosed herein can be implemented in hardware, software, firmware, or any combination thereof. In some embodiments, operations are carried out by one or more processors executing program instructions stored on one or more non transitory computer readable media. Such media can include, without limitation, semiconductor memory, magnetic storage, optical storage, and combinations thereof. Program instructions, when executed by the processors, cause the processors to perform the operations described herein.

Instructions can be delivered to computing devices in various ways, such as pre installation, physical distribution of media, or transmission over networks. Instructions received over a network can be stored in memory or persistent storage and then executed by one or more processors. Dedicated hardware logic, such as application specific integrated circuits or field programmable gate arrays, can be used alone or in combination with software to implement certain functionality.

Any methods described in connection with embodiments of the present disclosure can be represented as one or more flow diagrams or state diagrams. Blocks in such diagrams can correspond to modules, components, operations, or code segments that implement the associated functionality. Blocks can be reordered, combined, executed concurrently, or omitted according to implementation specific considerations, unless a particular ordering is required by the claims.

Examples and embodiments described herein illustrate, rather than limit, the claimed subject matter. Certain features have been described in connection with particular embodiments for clarity, but other embodiments can include such features in different combinations. Features described in separate embodiments can be combined, and features described in a single embodiment can be separated, unless such combinations or separations are inconsistent with the claims. The scope of the disclosure is defined by the claims and their equivalents.

### Overview

The present disclosure describes an evaluation of biomechanics and athletic performance, providing a multi-phase, multi-factor framework that may leverage adaptive AI models, 3D spatial data, and real-time feedback to improve form and performance. The methods and system disclosed herein may apply to key sports such as Basketball, Football, and Hockey, where precision in mechanics is critical for success. The system may identify phases, evaluate performance, and provide actionable insights to optimize athletic techniques.

The methods and systems may encompass a dynamic, AI-driven framework that evaluates performance across multiple phases using 3D spatial math and adaptive baselines. The above approach may offer precise, real-time insights that help athletes achieve optimal mechanics and continuously improve their performance.

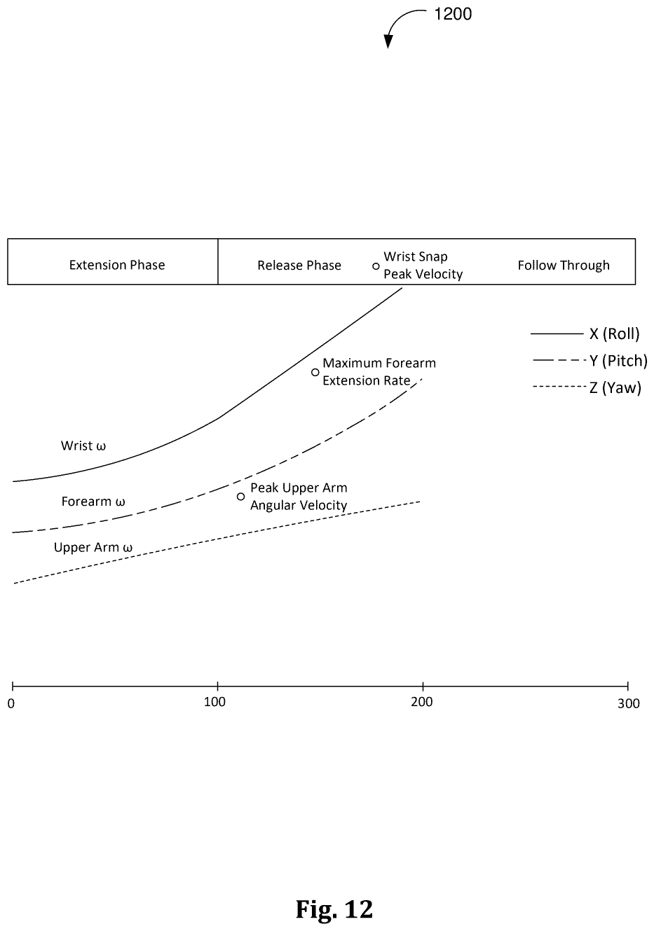

The methods and system may encompass a multi-factor evaluation framework to assess performance across distinct phases of an athlete's movement. Further, the disclosed method and system may apply dynamic AI models and customizable baselines to track, score, and improve mechanics over time. The present disclosure encompasses following key features:
1. Phase-Based Analysis: The methods and systems may evaluate biomechanical performance across distinct phases, such as the Pocket, Release, and Post Release phases of a basketball shot.
2. Adaptive Baselines: The methods and systems may support multiple baselines for comparing performance, including historical trends, injury recovery states, and hybrid coaching targets.
3. 3D Spatial Math Integration:
   The use of 3D spatial data, including quaternion-based representations, to measure joint angles, body alignment, and movement trajectories during biomechanical performance evaluation. Quaternions, or similar, enabling precise modeling of rotational motion by integrating a vector (representing the axis of rotation) and a scalar (representing the magnitude or angle of rotation), ensuring smooth, singularity-free computations in 3D space. The quaternion rotation dynamics of a wrist, a forearm and an upper arm of a player is illustrated in FIG. 12.
   Precision measurements may be based on 3D spatial data to ensure accurate evaluation of body mechanics without time-consuming camera calibration/re-calibration, as well as without requiring visual ‘assistance’ of other sensors or worn apparatus.
4. Gaussian-Based Scoring Models: A nuanced scoring system may capture deviations from ideal mechanics and track incremental improvements.
5. Real-Time Feedback: The methods and systems may provide adaptive feedback immediately, and may help athletes to correct mechanics and align with baseline targets.

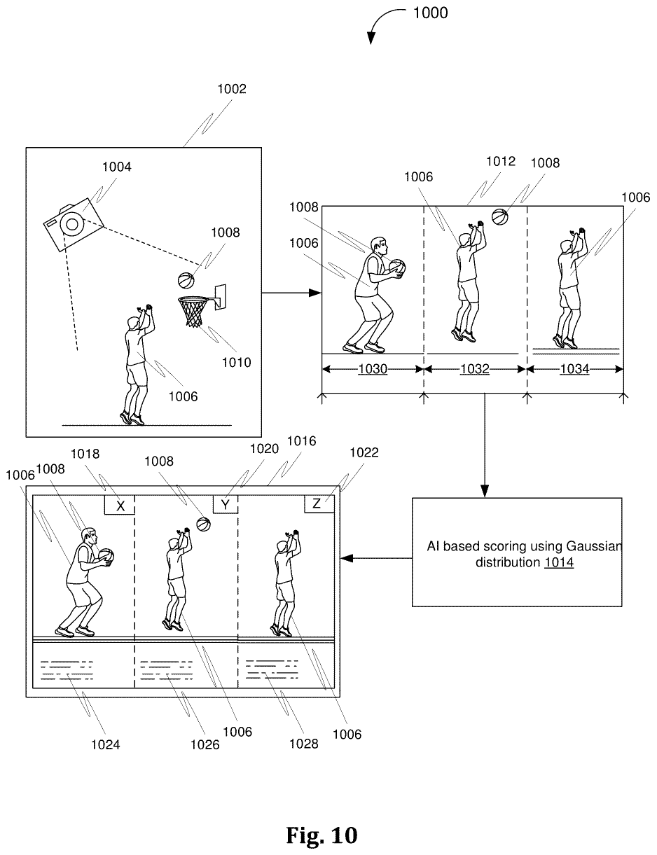

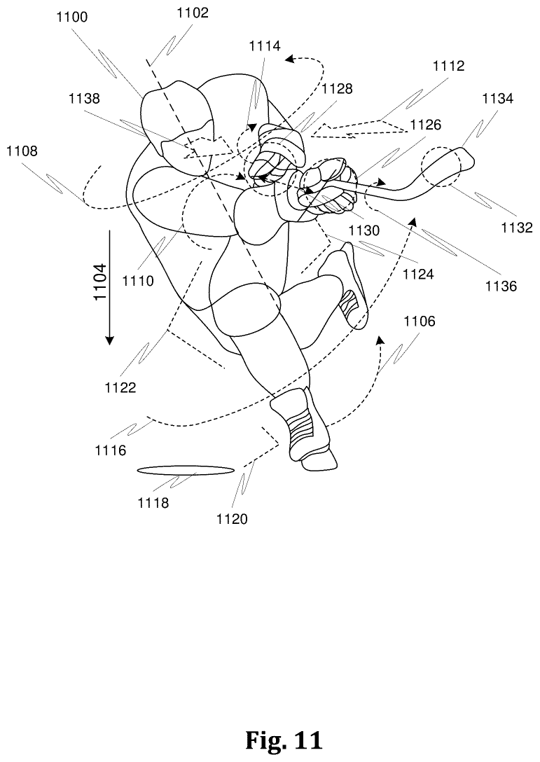

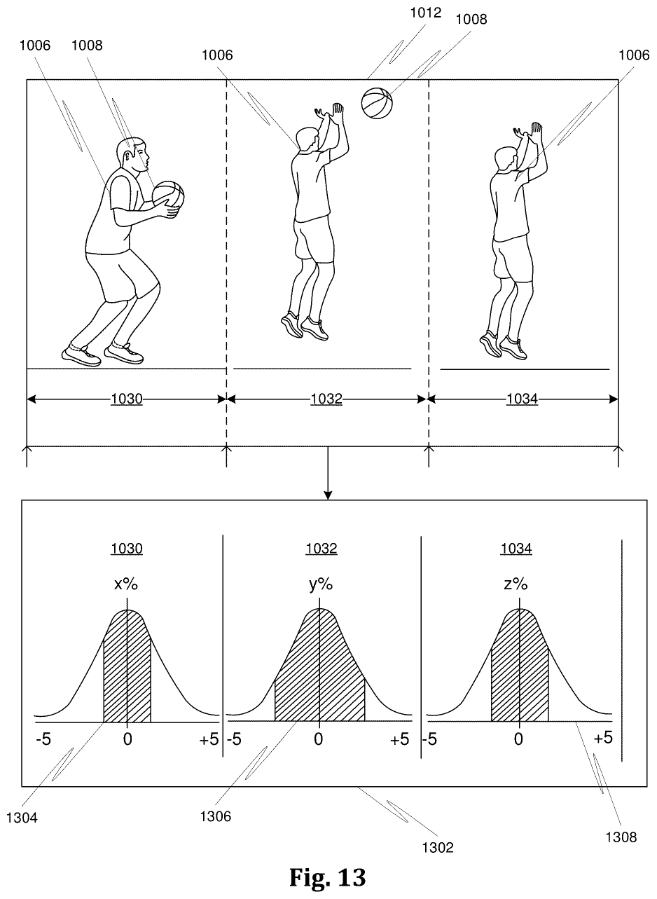

The following is the detailed description of each key feature:
1. Multi-Phase Evaluation Overview: The system may evaluate key performance phases across sports activities. For basketball, the system may break down a shot into the Pocket, Release, and Post Release phases.
   Pocket Phase: Preparing the shot by aligning the ball with the shooting shoulder.
   Release Phase: The ball leaves the player's hands, and wrist angles are measured for precision.
   Post Release Phase: The follow-through and balance are assessed to ensure consistent mechanics.
In football, the phases may be applied to quarterback throwing mechanics or lineman blocking, while in hockey, the phases may be applied to shooting or goal-tending actions. The multi-phase evaluation is illustrated in FIG. 10.
2. Adaptive Baseline Framework: The system may use customizable dynamic baselines to account for:
   Historical performance trends for tracking improvements.
   Injury recovery profiles with adjusted metrics reflecting recovery phases.
   Hybrid baselines may blend actual performance with coaching goals for targeted improvements.
   Scenario-based baselines for context-aware evaluations, such as comparing guarded vs. unguarded shots in basketball.
Further, athletes may be evaluated against one or more baselines simultaneously, providing a comprehensive view of performance trends and coaching alignment.
3. 3D Spatial Data Integration: Using 3D spatial mathematics, the system may precisely measure key biomechanical variables such as joint angles, body alignment, and movement trajectories. The spatial-temporal mechanics of a player in Hockey is illustrated in FIG. 11. The above approach may ensure accurate analysis across varying conditions without requiring time consuming camera calibration/re-calibration as well as without requiring visual ‘assistance’ of other sensors or worn apparatus.
4. Gaussian-Based Scoring System: The system may apply a Gaussian-based models to score performance based on how closely mechanics align with ideal metrics. The Gaussian-based scoring is illustrated in FIG. 13. Examples include:
   Evaluating wrist angles at 160 degrees ±5 degrees during a basketball shot.
   Measuring alignment during quarterback throws or hockey shots to assess consistency.
   Scoring balance and posture during football blocking actions or hockey saves.
Further, the Gaussian-based scoring method may capture incremental improvements and deviations, enabling precise tracking of progress over time.
5. Adaptive Feedback Mechanism
The feedback mechanism provides real-time, adaptive feedback based on baseline comparisons. For example:
   If a player is progressing toward a pre-injury baseline, positive reinforcement may be provided.
   Hybrid baselines may allow feedback to focus on both current performance and coaching recommendations.
   Feedback may be delivered through multi-modal information delivery mechanisms. The feedback may include video overlays, 3D reconstruction and audio as part of live training sessions via WebRTC (or similar) data channels (e.g. an audio whisper in the shooter earbuds as the shooter practice taking shots.
The adaptive feedback loop may ensure athletes receive actionable insights immediately, helping the athletes to adjust mechanics in real time.

### Key Aspects of the Disclosed System and Method

1. Multi-Phase Evaluation System: A system may evaluate athletic performance across distinct phases using adaptive AI models and customizable baselines to assess biomechanics.
2. Adaptive Baseline Framework: A method may create and apply multiple dynamic baselines, including historical performance trends, injury recovery profiles, hybrid coaching targets, and scenario-based conditions, to guide performance evaluation and feedback.
3. 3D Spatial Math Integration: The use of 3D spatial data, including quaternion-based representations, to measure joint angles, body alignment, and movement trajectories during biomechanical performance evaluation. Quaternions, or similar, enable modeling of rotational motion by integrating a vector (representing the axis of rotation) and a scalar (representing the magnitude or angle of rotation), ensuring smooth, singularity-free computations in 3D space.
4. Gaussian-Based Scoring Method:
A scoring system may use Gaussian distributions to measure deviations from ideal performance metrics across multiple phases.
5. Adaptive Feedback System:
A feedback mechanism may adjust insights based on real-time and historical comparisons with dynamic baselines and evolving performance trends.

The methods and systems may ensure continuous improvement and personalized coaching, making the methods and systems highly adaptable for various sports applications.

The method and system may analyze sports equipment (e.g., balls, hockey sticks, baseball bats, etc.) in addition to the biomechanical data for human motion. The sports equipment is as integral to athletic performance as the athlete's biomechanics, yet existing systems may not capture the analyzing of sports equipment. For instance:
Golf: While conventional systems visualize body key points, the conventional systems may fail to track and represent the club and the club's interaction with the swing plane.
Hockey: The stick's position and interaction with the puck are critical for shot accuracy and passing mechanics.
Basketball: Tracking both the ball's trajectory and player motion allows for comprehensive analysis of dribbling, shooting, and passing mechanics.

The following are the key features of the disclosed system:
1. Equipment Integration in 3D Models: The disclosed system may incorporate sports equipment into 3D spatial analyses, ensuring the sport equipment is represented as part of the biomechanical and situational evaluations. Quaternions or similar methods may track rotations and interactions between the athlete and equipment.
2. Composite Performance Metrics: The disclosed system may develop metrics that integrate athlete and equipment data (for example, the alignment between a player's hand and a basketball during release or the angle of a hockey stick blade during a slap shot).
3. Dynamic Equipment Modeling: The dynamic equipment modelling may allow for adaptable modeling of different equipment types, enabling evaluations across multiple sports contexts.
4. Feedback and Instruction Enhancements: The disclosed system may provide real-time feedback that includes both the athlete and equipment (for example, suggesting grip adjustments or highlighting inconsistencies in equipment alignment)
The system may combine rules-based methodologies (heuristics) with the adaptive AI models to deliver real-time feedback. Using 3D spatial math and Gaussian-based scoring models, the system may evaluate key metrics to provide precise, actionable insights for athletes, coaches, and trainers. The framework may also support adaptive baselines to track progress over time and offer personalized evaluations based on prior performance, recovery states, and hybrid coaching goals.

The present disclosure may address multiple sports contexts, including basketball, football, and hockey. The system may emphasize the need to evaluate mechanics based on sport-specific phases, with each activity having distinct phases tailored to the motion (e.g., throwing mechanics in football, shot release in hockey). The flexible baseline framework may allow athletes to be evaluated against various performance goals and states, ensuring relevance over time.

In some embodiments, the present disclosure may provide inherent technical improvements in the way biomechanical performance of a physical activity is evaluated and fed back to a user. In some embodiments, the technical improvements may relate to a multi-phase, AI-driven analysis of a content stream data, a three-dimensional spatial representation of motion using quaternion-based modeling, a Gaussian-based scoring mechanism, an adaptive baseline framework, context-sensitive evaluation, integrated modeling of a sport object and a player, real-time communication of feedback, hybrid AI and rule-based reasoning, and longitudinal storage and reuse of feedback data.

In some embodiments, a first inherent technical improvement may be directed to a multi-phase AI analysis of a content stream data that may be associated with a physical activity. In some embodiments, existing systems may treat a recorded motion as a single undifferentiated segment and may therefore be unable to distinguish between preparatory, execution, and follow-through portions of a movement. In some embodiments, the treating of the recorded motion as the single undifferentiated segment may lead to a technical problem where error localization in time is poor and where model training may be less accurate because features from different phases may be entangled.

In some embodiments, the present disclosure may address the above technical problem by causing a processing device to segment the content stream data into two or more time intervals corresponding to two or more phases of the physical activity, and to determine one or more characteristic data for each phase. In some embodiments, a basketball shot may be segmented into a pre-release phase, a release phase, and a post-release phase, and a separate characteristic data may be generated for each of these phases. In some embodiments, for a football throw, a wind-up phase, an acceleration phase, and a follow-through phase may be used. In some embodiments, the AI model may be conditioned on a phase label, and a separate sub-model or head of a neural network may be used for each phase, thereby improving temporal resolution and reducing interference between phases. In some embodiments, the technology being improved may be a motion analysis engine that may operate on time-series image or sensor data, and the improvement may arise from phase-specific feature extraction and phase-specific error detection.

In some embodiments, the multi-phase AI analysis may be implemented in different ways. In some embodiments, the processing device may perform a temporal segmentation algorithm based on changes in joint velocity, hand-ball distance, or stick-puck distance, and may assign each frame of the content stream data to a phase. In some embodiments, a recurrent neural network or transformer model may output phase probabilities per frame, and a phase boundary may be identified by thresholding the phase probabilities.

In some embodiments, a finite-state machine may be driven by the AI model scores to transition from pocket to release to post-release states. In some embodiments, once the phases are identified, the AI model may compute characteristic data such as joint angle at release, center-of-mass shift during pre-load, or follow-through direction, all tied to precise time intervals. In some embodiments, the disclosed system may directly improve a temporal modeling technology used in computer vision-based sports analytics.

In some embodiments, a second inherent technical improvement may be directed to quaternion-based three-dimensional spatial modeling that may be performed without time-consuming camera calibration or external sensor attachment. In some embodiments, conventional optical motion capture may rely on multi-camera calibration or body-worn markers, which may be cumbersome and may not function well in unconstrained environments such as public courts. In some embodiments, the relying on the multi-camera calibration or body-worn markers may lead to a technical problem of brittle or non-portable biomechanics analysis.

In some embodiments, the present disclosure may address the technical problem of brittle or non-portable biomechanics analysis by causing the processing device to analyze the content stream data based on a three-dimensional spatial data that may include quaternions representing rotational orientation of body parts and, in some embodiments, a sport object. In some embodiments, a quaternion may encode an axis of rotation as a vector component and an angle of rotation as a scalar component, thereby avoiding gimbal lock and providing smooth interpolation. In some embodiments, the technology being improved may be a three-dimensional pose representation technology for biomechanical analysis.

In some embodiments, the quaternion-based three-dimensional spatial data may be derived from monocular video using a learned pose estimation model that may output joint orientations directly as quaternions. In some embodiments, the processing device may compute relative orientation between body segments, for example, a quaternion representing the rotation of a forearm with respect to an upper arm, and may derive joint angle and angular velocity data from the relative orientation. In some embodiments, the system may compute orientation difference between a target reference quaternion and a measured quaternion using quaternion multiplication and inverse operations, and may thereby produce a precise deviation measure.

In some embodiments, the precise deviation measure may be used to measure whether a shooter's wrist is at an ideal release orientation. In some embodiments, multiple cameras may be used when available, and the processing device may fuse independent quaternion estimations to improve robustness; however, in some embodiments, the system may be able to function with a single camera due to the learned mapping from image space to quaternion space, thereby reducing the calibration requirement.

In some embodiments, a third inherent technical improvement may be directed to a Gaussian-based scoring mechanism that may use statistical deviations from a reference metric to generate a score data. In some embodiments, existing scoring systems may use threshold-based or coarse binning schemes, which may not capture fine-grained improvements or may be sensitive to noise. In some embodiments, the usage of the threshold-based or coarse binning schemes may present a technical problem of low resolution and poor robustness in scoring. In some embodiments, the present disclosure may address the technical problem of low resolution and poor robustness by causing the processing device to generate the score data based on the deviation data and a Gaussian distribution associated with the reference metric.

In some embodiments, a reference metric may include an ideal wrist angle of 160 degrees with an allowable variation of ±5 degrees, and the processing device may treat the distribution of acceptable angles as a Gaussian with a mean and a standard deviation. In some embodiments, the measured metric may then be transformed into a normalized score based on the probability density under the Gaussian distribution. In some embodiments, multiple metrics may be combined using weighted Gaussian-based scores to produce an overall performance index. In some embodiments, the technology being improved may be a scoring engine that may convert raw kinematic metrics into interpretable numerical scores.

In some embodiments, the Gaussian-based scoring mechanism may be implemented using precomputed Gaussian parameters derived from expert data, or may be learned dynamically from historical data of a user. In some embodiments, the system may fit a Gaussian distribution to a cluster of “successful” events, such as made shots, and may update mean and variance based on new successful samples. In some embodiments, separate Gaussian distributions may be maintained per phase, such as a distribution for pre-release alignment and a distribution for release wrist angle.

In some embodiments, the processing device may determine percentile-like measures, such as how many standard deviations away from the ideal a particular metric may be, and may use the percentile-like measures to generate feedback data that may characterize the magnitude of deviation.

In some embodiments, a fourth inherent technical improvement may be directed to an adaptive baseline framework that may account for historical performance trends, injury recovery profiles, hybrid coaching targets, and scenario-based baselines. In some embodiments, existing systems may use static, global baselines that may not reflect an individual's evolving condition. In some embodiments, the use of static and global baselines may lead to a technical problem in which feedback may be misaligned with a user's current capability or training stage.

In some embodiments, the present disclosure may address the technical problem of misalignment of the feedback with user's current capability or training stage by causing the processing device to generate an adaptive baseline data based on feedback data stored by a storage device and feedback data from prior physical activities. In some embodiments, the system may maintain a pre-injury baseline, an injury-recovery baseline, and a target coaching baseline, and may select or interpolate between these baselines depending on context data. In some embodiments, the technology being improved may be a personalization and baseline-management technology used in performance evaluation engines.

In some embodiments, the adaptive baseline framework may be implemented by computing rolling averages or exponentially decayed averages of metric data over a configurable time window. In some embodiments, the system may tag each historical performance with a state label such as “pre-injury” or “recovery,” and may maintain separate baselines for each label. In some embodiments, a coach may supply a target metric set, and the processing device may generate a hybrid baseline data that may combine the actual historical metrics with the coach-specified targets, such as by weighting the actual historical metrics with the coach-specified targets according to a progression schedule. In some embodiments, the adaptive baseline may be scenario dependent, and the context data may indicate whether a shot is guarded or unguarded, allowing the system to maintain distinct reference metrics for each scenario.

In some embodiments, a fifth inherent technical improvement may be directed to context-sensitive evaluation of the physical activity based on context data that may be identified from the content stream data. In some embodiments, conventional systems may provide a single evaluation irrespective of game context, thereby creating a technical problem in distinguishing whether deviations are acceptable under defensive pressure. In some embodiments, the present disclosure may address the technical problem in the distinguishing of deviations by causing the processing device to analyze the content stream data to determine a contextual variable, such as distance to a defender or whether a shot is contested, and to identify a context data based on the contextual variable. In some embodiments, the processing device may generate a target metric based on the context data and the reference metric, thereby allowing adjusted expectations under different play conditions. In some embodiments, the technology being improved may be a context-aware analytics component for sports performance systems.

In some embodiments, the context-sensitive evaluation may be implemented by detecting additional objects or agents in the content stream data, such as a defender in basketball or an opposing player in hockey, and computing relative positions. In some embodiments, a threshold on defender proximity may classify a shot as guarded. In some embodiments, the AI model may also detect whether a shot occurs off the dribble or off a catch, and the context data may encode the state. In some embodiments, the system may use separate Gaussian-based scoring parameters for guarded and unguarded conditions, or may vary weights of metrics like shot speed and release angle depending on context.

In some embodiments, a sixth inherent technical improvement may be directed to integrated analysis of a player characteristic and a sport object characteristic within the same three-dimensional spatial framework. In some embodiments, conventional biomechanics systems may track only body pose while ignoring the motion of the ball, stick, club, or other sport object, which may create a technical problem where key aspects of ball-hand interaction or stick-puck interaction are unmodeled. In some embodiments, the present disclosure may address the technical problem of lack of modeling of ball-hand interaction or stick-puck interaction by causing the processing device to analyze movement trajectory of a player and movement trajectory of a sport object and to generate metrics that may capture their alignment or timing relationships. In some embodiments, the technology being improved may be a coupled human- equipment motion modeling technology.

In some embodiments, the integrated analysis of a player and a sport object may be implemented by estimating a three-dimensional trajectory of a ball in basketball based on the ball's pixel location and camera model, and comparing this trajectory to the orientation and path of the shooter's hand. In some embodiments, the system may compute a release timing metric that may measure the delay between peak upward motion of the elbow and ball release. In some embodiments, for hockey, the system may track the stick blade orientation and the puck trajectory, and may compute a metric representing consistency of blade-puck contact angle across shots. In some embodiments, the integrated modeling may be directly represented using quaternions for both body segments and sport objects, allowing relative orientation computations between hand and ball or between stick and puck.

In some embodiments, a seventh inherent technical improvement may be directed to real-time feedback transmission using a real-time communication protocol such as WebRTC or a similar protocol. In some embodiments, existing systems may process video offline and deliver feedback after a delay, which may result in a technical problem of poor responsiveness and limited applicability in live training. In some embodiments, the present disclosure may address the problem of poor responsiveness and limited applicability in live training by causing the processing device to generate a formatted feedback data suitable for real-time transmission and by causing the communication device to transmit the formatted feedback data based on a real-time communication protocol.

In some embodiments, the formatted feedback data may encode metrics, score data, and narrative statements in a compact representation that may be overlaid on a video stream by a client device. In some embodiments, the technology being improved may be a low-latency streaming and real-time feedback technology for sports analytics.

In some embodiments, the real-time feedback pipeline may be implemented by processing the content stream data in sliding windows and generating feedback data for each completed phase as soon as enough frames are buffered. In some embodiments, the server-side processing device may encode feedback messages as JSON or binary messages and may send the feedback messages over data channels of a WebRTC session. In some embodiments, the system may maintain a strict processing budget per frame, for example by using a lightweight AI model at inference time, so that end-to-end latency between motion and feedback may remain within a target threshold suitable for near-immediate coaching.

In some embodiments, an eighth inherent technical improvement may be directed to hybrid use of an AI model and a rule-based algorithm to generate feedback data. In some embodiments, purely data-driven models may produce opaque outputs, and purely rule-based systems may lack adaptability, leading to a technical problem of either low explainability or low adaptability. In some embodiments, the present disclosure may address the technical problem of either low explainability or low adaptability by allowing the processing device to generate metrics using the AI model, and then to apply a heuristic algorithm to the metrics to generate feedback data that may be interpretable and consistent.

In some embodiments, the rule-based algorithm may encapsulate coaching heuristics such as “if wrist angle deviation is above a threshold and shot success rate is below a threshold, recommend reducing wrist flexion.” In some embodiments, the technology being improved may be an explainable feedback generation engine that may combine machine-learned analysis with deterministic reasoning.

In some embodiments, the hybrid AI and rule-based system may be implemented by mapping AI-generated metrics and scores into discrete states, and then applying rule sets that may be defined by coaches or learned from data. In some embodiments, the rule-based layer may also serve as a safety filter to prevent unstable or contradictory feedback when AI model confidence is low. In some embodiments, the system may store rule triggers and corresponding feedback messages in the storage device, and may update the rules through a configuration interface without retraining the AI model.

In some embodiments, a ninth inherent technical improvement may be directed to longitudinal storage and reuse of feedback data, metric data, and associated context data. In some embodiments, conventional systems may store raw videos without structured histories of metric trends, which may lead to a technical problem of inefficient retrieval and limited support for progression modeling. In some embodiments, the present disclosure may address the technical problem of inefficient retrieval and limited support for progression modeling by causing the storage device to store feedback data, processed characteristic data, deviation data, score data, and context data in relation to time and session identifiers.

In some embodiments, the structured storage may enable the processing device to retrieve historical sequences for generating adaptive baseline data, computing trends, and training updated AI models. In some embodiments, the technology being improved may be a time-series data management layer specialized for biomechanics and sports performance analytics.

In some embodiments, the longitudinal storage may be implemented using a schema that may include per-session records with timestamps, per-phase metric records, and per-context labels, enabling efficient queries such as “retrieve last 100 release wrist angle measurements under unguarded context.” In some embodiments, the system may maintain index structures for fast retrieval by user, sport, context, or phase. In some embodiments, the processing device may run periodic batch analytics using the stored data to compute trendlines or to recompute adaptive baselines.

In some embodiments, the present disclosure may further encompass additional technical improvements that may be added to the system and method, while remaining consistent with the overall architecture of a communication device, a processing device, and a storage device. In some embodiments, the additional features may improve specific technologies such as representation learning for motion data, model generalization, distributed learning, latency optimization, and uncertainty-aware feedback.

In some embodiments, a first additional technical improvement may be directed to self-supervised representation learning on unlabeled sports content stream data. In some embodiments, a technical problem may arise because labeled biomechanical data may be scarce and expensive to annotate, leading to limited generalization of the AI model across sports, body types, and camera viewpoints. In some embodiments, the processing device may be configured to perform self-supervised learning tasks such as predicting future frames, reconstructing masked joint trajectories, or distinguishing temporally shuffled sequences, using the content stream data stored by the storage device. In some embodiments, the technology being improved may be a motion representation learning technology for sports analytics.

In some embodiments, the self-supervised representation learning may be implemented by training an encoder network that may map raw video or pose sequences into an embedding space, where the training signal may be derived from contrastive objectives or reconstruction objectives rather than explicit labels. In some embodiments, the system may pretrain the encoder on a large corpus of unlabelled content stream data from various sports and later fine-tune the encoder on a smaller labeled dataset for computing characteristic data and metrics. In some embodiments, the encoder may be shared across different sports, while the final task heads may be sport-specific. In some embodiments, the storage device may maintain separate datasets for pretraining and fine-tuning, and the processing device may switch between modes based on configuration.

In some embodiments, a second additional technical improvement may be directed to graph-based or topology-aware modeling of biomechanical structures. In some embodiments, conventional models may treat joint data as a flat feature vector, ignoring the kinematic tree structure of the body, which may lead to a technical problem of suboptimal learning of inter-joint dependencies. In some embodiments, the processing device may implement a graph neural network that may treat joints as nodes and bones as edges, thereby exploiting the structure of the human body and, in some embodiments, the structure of a sports object such as a stick. In some embodiments, the technology being improved may be a structured neural modeling technology for biomechanical data.

In some embodiments, this graph-based modeling may be implemented by constructing an adjacency matrix that may represent connectivity between joints, and by applying message-passing operations along edges to propagate information about angular relationships and forces. In some embodiments, the processing device may extend the graph to include additional nodes for the ball, puck, or club, connected to hand or foot nodes, thereby modeling human-equipment interactions as part of the same graph. In some embodiments, temporal dynamics may be incorporated by stacking graph layers across time or by combining graph convolution with recurrent layers.

In some embodiments, a third additional technical improvement may be directed to an edge-cloud collaboration strategy for latency reduction and bandwidth optimization. In some embodiments, a technical problem may arise when high-resolution content stream data is transmitted to a cloud server, causing bandwidth overhead and latency that may impair real-time feedback. In some embodiments, the system may be configured such that some pre-processing may be performed by an edge node, while the main AI inference and feedback generation may be performed by the remote processing device. In some embodiments, the technology being improved may be an edge-cloud distributed processing technology in the context of real-time sports analytics.

In some embodiments, the edge-cloud collaboration may be implemented by having a client device or a nearby gateway produce a compressed pose representation, such as a set of two-dimensional keypoints or low-resolution heatmaps, and transmitting only the reduced representation as the content stream data to the communication device. In some embodiments, the processing device in the cloud may then reconstruct three-dimensional spatial data and perform advanced analysis. In some embodiments, model distillation may be used such that a smaller version of the AI model may run on the edge while a larger model may run in the cloud; the smaller model may provide quick, approximate feedback while the cloud model may refine the feedback after a short delay.

In some embodiments, a fourth additional technical improvement may be directed to federated learning for model update without centralizing raw athlete data. In some embodiments, a technical problem may arise from data privacy and regulatory constraints that may prevent raw video data from being aggregated, limiting the scope of model improvement. In some embodiments, the system may support a process in which local instances of the AI model may be trained on-device or on-premise with local content stream data, and only model updates or gradients may be transmitted to a central server for aggregation by a processing device. In some embodiments, the technology being improved may be a privacy-preserving learning and model-update technology.

In some embodiments, the federated learning pipeline may be implemented such that the communication device may periodically receive model update messages from a set of client installations, and the processing device may aggregate the updates using weighted averaging or more advanced optimization techniques. In some embodiments, the aggregated model parameters may then be redistributed to the clients. In some embodiments, the storage device may maintain versioned models and associated metadata, allowing rollback or per-cohort customization.

In some embodiments, a fifth additional technical improvement may be directed to uncertainty-aware feedback generation. In some embodiments, conventional systems may return a single deterministic feedback even when input data quality is poor, leading to a technical problem of overconfident but unreliable guidance. In some embodiments, the processing device may compute an uncertainty measure associated with the characteristic data, metric data, or score data, for example, by using an ensemble of models or a Bayesian approximate inference technique. In some embodiments, the technology being improved may be a confidence-aware inference and feedback mechanism.

In some embodiments, the uncertainty-aware feedback may be implemented such that the processing device may generate both a point estimate and an uncertainty value (such as variance or entropy) for each metric. In some embodiments, the heuristic algorithm may take into account the uncertainty when deciding which feedback messages to issue, for example, by suppressing specific prescriptive advice when uncertainty exceeds a threshold and instead flagging the need for additional samples or better camera placement. In some embodiments, the feedback data transmitted by the communication device may thus encode not only a score but also an indication of confidence, allowing downstream visualization systems to render appropriate cues such as “low confidence” warnings.

Further, the present disclosure describes a method of facilitating performance evaluation of physical activities. Further, the facilitating performance evaluation of physical activities is and/or includes facilitating performance evaluation of users performing physical activities.

Further, in some embodiments, the method may include obtaining, using the processing device, at least one output using at least one large language model based on at least one input and at least one instruction. Further, the generating of the feedback data may be further based on the at least one output. Further, the at least one large language model may comprise a transformer-based autoregressive sequence generation model including a plurality of transformer decoder layers configured to generate an output token sequence conditioned on an input context.

Further, in some embodiments, the at least one input may include (i) the at least one content and/or the at least one characteristic data determined based on the at least one content data using at least one AI model, (ii) at least one time-window identifier associated with performance of the physical activity, and (iii) at least one device characteristic corresponding to at least one of a capability, limitation, operation, and state of the at least one user device. Further, the at least one device characteristic may be obtained from an operating system application programming interface (API) of the at least one user device, a network stack measurement routine, a device capability profile stored in memory, or a telemetry message transmitted from the at least one user device to the processing device.

Further, in some embodiments, the at least one instruction may specify at least (i) a required machine-readable output format for a spatio-temporal feedback instruction set and (ii) at least one constraint derived from at least one dynamic user characteristic. Further, the at least one constraint may include a maximum allowed joint-angle correction per time window, a maximum haptic intensity level, a maximum feedback update frequency, a minimum confidence threshold before issuing real-time corrective actions, or a requirement to generate feedback in a selected modality that the at least one user device supports.

Further, in some embodiments, the obtaining of the at least one output may include determining, using the processing device, at least one value for at least one model parameter associated with the at least one large language model based on the at least one instruction and/or at least one dynamic user characteristic. Further, the at least one model parameter may govern inference behavior and may include a maximum output token budget, a context-window length, a decoding parameter (including temperature, top-k sampling, top-p sampling, beam size, or repetition penalty), and/or an execution parameter including numerical precision or quantization bit-width for executing model weights.

Further, in some embodiments, the obtaining of the at least one output may include selecting, using the processing device, an execution configuration to satisfy a target resource constraint. Further, the execution configuration may include selecting between (i) a first precision configuration and (ii) a second reduced-precision configuration, selecting between local execution and edge execution, and/or selecting a model variant having a reduced parameter count. Further, the target resource constraint may include a target end-to-end latency, a memory ceiling, and/or a maximum network payload size for each incremental update.

Further, in some embodiments, the method may include inputting the at least one input to the at least one large language model based on the determining and/or the selecting. Further, “inputting” may include constructing a composite model context comprising an instruction portion and an input portion, encoding the composite model context into model-consumable inputs, and providing the model-consumable inputs to the transformer-based autoregressive sequence generation model for inference.

Further, in some embodiments, inputting the at least one input and the at least one instruction to the at least one large language model may include the following steps:
1. Further, the processing device may serialize at least a portion of the at least one characteristic data for the time-window identifier into a structured representation, including time-indexed feature values (e.g., orientation quaternion values, joint angle values, deviation metrics, and associated confidence values).
2. Further, the processing device may construct an instruction payload specifying an output schema and constraints, including at least one of a maximum number of control primitives per time window, a permitted modality set determined from device characteristics, and a maximum payload size per update.
3. Further, the processing device may combine the instruction payload with the structured representation and the user query to generate a composite model context.
4. Further, the processing device may encode the composite model context into model-consumable inputs by tokenizing text portions into token identifiers and converting numeric portions into a canonical text form or a numeric embedding representation.
5. Further, the processing device may provide the token identifiers and/or embeddings to the transformer-based autoregressive sequence generation model along with inference configuration values corresponding to the determined model parameters.
6. Further, the transformer-based autoregressive sequence generation model may generate a structured output token sequence, which is decoded into the machine-readable spatio-temporal feedback instruction set. Further, the feedback data comprises the machine-readable spatio-temporal feedback instruction set.

Further, in some embodiments, the at least one output may include a machine-readable spatio-temporal feedback instruction set that specifies time-indexed feedback actions executable by the user device. Further, the feedback data is and/or include the machine-readable spatio-temporal feedback instruction set. Further, the machine-readable instruction set may include a plurality of time-indexed control primitives, and each control primitive may include fields including (i) a time offset, (ii) a modality identifier, (iii) an action type identifier, and (iv) one or more action parameters.

Further, in some embodiments, the action parameters may include a corrective rotation vector, a corrective translation vector, a target pose parameter, a haptic waveform identifier, a haptic intensity, an audio prompt identifier, or a visual overlay identifier. Further, the corrective rotation vector may be represented as Euler deltas or quaternion deltas (e.g., Δq) depending on the selected schema.

Further, in some embodiments, the method may include validating, using the processing device, the machine-readable spatio-temporal feedback instruction set prior to transmitting the feedback data. Further, validating may include checking that required fields are present, checking type conformance, checking bounds on numeric values, and verifying that time offsets fall within the referenced time window.

Further, in some embodiments, in response to detecting that at least one control primitive violates the schema, the processing device may correct the control primitive, remove the control primitive, or re-invoke inference with additional constraints. Further, schema validation enables deterministic parsing and execution on the user device and reduces failure modes caused by malformed outputs.

Further, in some embodiments, the method may include selecting a numerical precision or quantization bit-width for executing model weights based on the device characteristic. Further, the method may execute the model using at least one of 16-bit, 8-bit, 4-bit, or mixed-precision inference to satisfy memory and latency constraints.

Further, in some embodiments, the method may include selecting between a first model variant and a second compressed model variant. Further, the second compressed model variant may be produced using quantization, pruning, knowledge distillation, or low-rank adaptation. Further, selecting among model variants and precision configurations reduces memory footprint and improves inference latency, thereby improving computing performance for real-time feedback generation.

Further, in some embodiments, the method may include selecting between local execution and edge execution based on measured network characteristics and device state. Further, the processing device may measure network latency and bandwidth using round-trip time measurements, transport-layer statistics, or application-layer telemetry. Further, in response to determining that local compute is insufficient or that latency targets cannot be satisfied locally, the method may route inference to an edge compute node within a threshold network distance of the user device, thereby reducing end-to-end delay.

Further, in some embodiments, the method may include transmitting the feedback data as a sequence of incremental updates, each incremental update corresponding to a time window and including a sequence identifier and time offset. Further, the user device may maintain a jitter buffer to reorder out-of-order updates and to smooth variable arrival times.

Further, in some embodiments, the method may include dynamically adjusting jitter buffer depth based on measured latency variation. Further, adjusting the jitter buffer depth may include increasing the buffer depth when network jitter increases and reducing the buffer depth when network jitter decreases, thereby maintaining a target end-to-end feedback delay while reducing dropped or late feedback updates.

Further, in some embodiments, the method may include applying forward error correction or redundant packetization to incremental updates based on measured packet loss rate, thereby improving continuity of spatio-temporal feedback actions under adverse network conditions.

Further, in some embodiments, the method may include computing a confidence score associated with the spatial characteristic data based on sensor noise metrics, occlusion detection, and/or residual error of the spatial orientation model. Further, the confidence score may be computed per time window or per feature.

Further, in some embodiments, the method may select a feedback modality, intensity, or update rate based on the confidence score. For example, when confidence is below a threshold, the method may reduce update frequency, switch from real-time haptics to summary visual guidance, or increase smoothing of corrective actions. This reduces erroneous or unstable feedback outputs caused by low-quality sensor estimates.

Further, in some embodiments, the method may include enforcing device constraints prior to transmitting or executing the machine-readable instruction set. Further, enforcing may include verifying that haptic intensity does not exceed a maximum supported by the device, that audio amplitude does not exceed a maximum level, that visual overlay density does not exceed a rendering threshold, and that update rate does not exceed a maximum supported frame rate. Further, in response to a violation, the method may clamp values, remove control primitives, or re-generate the instruction set under stricter constraints.

Further, in some embodiments, the at least one content data may include at least one of (i) a video stream of the at least one physical activity and (ii) a time-series sensor stream captured during the at least one physical activity. Further, the video stream may include a plurality of frames with associated timestamps, and the time-series sensor stream may include samples organized into time windows (e.g., overlapping windows) aligned to the timestamps. Further, the at least one content data may be associated with data characteristics comprising a sampling rate and a timestamp or timecode. Further, the data characteristics may include a device identifier of the at least one content source device, such that device-specific correction parameters and/or domain-adaptation parameters may be selected.

Further, in some embodiments, the at least one calibration parameter set may include at least one of camera intrinsics (e.g., focal length, principal point, distortion coefficients), camera extrinsics (e.g., orientation), sensor bias/scale factors (e.g., accelerometer bias and gyroscope bias), coordinate-frame definitions, and/or time synchronization offsets between sensors. Further, the noise characteristic may include at least one of signal-to-noise ratio, variance, drift, jitter, or compression artifact level. Further, compression artifact level may be computed from at least one of a compression format identifier, quantization parameters, blockiness metrics, and/or a bit-rate metric, thereby providing objective, repeatable measures that can be consumed by the AI model and/or the normalization pipeline.

Further, in some embodiments, the method may include normalizing the at least one content data prior to inputting the at least one content data to the at least one AI model. Further, the normalizing may include applying at least one calibration and correction operation selected from lens-distortion correction based on camera intrinsics, coordinate-frame transformation based on the at least one calibration parameter set, temporal resampling to a target sampling rate, and denoising based on the noise characteristic.

Further, in some embodiments, lens-distortion correction may be performed by mapping each pixel coordinate through an inverse distortion model parameterized by the distortion coefficients stored in the calibration parameter set. Further, coordinate-frame transformation may include applying a rotation matrix and translation vector to transform sensor measurements from a device coordinate frame into a reference coordinate frame used by the reference data. Further, temporal resampling may include interpolation (e.g., linear interpolation or spline interpolation) to map irregular samples to the target sampling rate, and may further include aligning sensor timestamps to video timestamps using a time offset stored in the calibration parameter set. Further, denoising may include applying at least one of a low-pass filter, a Kalman filter, a median filter, or a frequency-domain attenuation operation, where filter parameters are selected based on the noise characteristic.

Further, in some embodiments, the at least one AI model may comprise a feature-extraction network configured to output (i) a content feature representation derived from the at least one content data and (ii) a reference feature representation derived from the at least one reference data. Further, the feature-extraction network may include one or more convolutional layers, transformer encoder layers, recurrent layers, and/or fully connected layers configured to process spatiotemporal inputs.

Further, in some embodiments, determining the at least one characteristic data may further comprise computing a feature-discrepancy metric between the content feature representation and the reference feature representation. Further, the feature-discrepancy metric may be computed over matched time windows of the physical activity such that a window of content features is compared to a corresponding window of reference features (e.g., aligned by detected gait cycle events or swing phases). Further, determining the at least one characteristic data may further comprise dynamically varying model parameters of the feature-extraction network during inference for the at least one physical activity based on the feature-discrepancy metric and at least one of the data characteristics. Further, the at least one characteristic data may further comprise the at least one actual metric (e.g., joint angle, cadence, stride length) computed from the aligned feature representations.

Further, in some embodiments, the dynamically varying may be performed as a domain adaptation procedure in which: (i) baseline model parameters are loaded from memory; (ii) a candidate update to a permitted subset of parameters is computed using the feature-discrepancy metric; (iii) the candidate update is accepted when the feature-discrepancy metric decreases; and (iv) the procedure repeats until the feature-discrepancy metric satisfies a threshold such that the content feature representation is characteristically aligned with the reference feature representation for generation of the feedback data. Further, the threshold may be selected based on at least one of the activity type, the device identifier, and/or the noise characteristic, thereby providing an objective criterion for convergence and alignment. The dynamic variation is not merely “optimizing a model,” but compensates for device-dependent and capture-dependent distortions indicated by the data characteristics, improving the reliability of biomechanical metrics derived from heterogeneous sensors.

Further, in some embodiments, dynamically varying the model parameters may include selectively updating only a domain-adaptation subset of the model parameters that includes at least one of batch-normalization affine parameters, a feature-scaling vector, an attention-weight subset, or an adapter-layer weight matrix, while constraining remaining weights of the feature-extraction network to remain fixed, thereby compensating for device-dependent variation represented by the data characteristics.

Further, in some embodiments, the domain-adaptation subset may be explicitly identified in memory as a parameter index set or named layer set such that the system updates only the identified parameters during inference. Further, constraining remaining weights may include setting gradients to zero for non-adaptation parameters and/or preventing writes to non-adaptation parameters, thereby ensuring bounded behavior and repeatability. Further, updating batch-normalization affine parameters may include adapting scale and shift values to normalize features under different noise and sampling conditions. Further, updating an adapter-layer weight matrix may include updating a low-rank projection used to map content features into a canonical feature space used by the reference data.

Further, in some embodiments, the feature-discrepancy metric may comprise at least one of cosine distance, Mahalanobis distance, maximum mean discrepancy, or Kullback-Leibler divergence. Further, dynamically varying the model parameters may comprise minimizing, over a bounded number of iterations, a loss function that includes (i) the feature-discrepancy metric and (ii) a regularization term that bounds deviation of the updated model parameters from baseline model parameters stored in memory.

Further, in some embodiments, the bounded number of iterations may be selected to satisfy a latency constraint for real-time feedback generation (e.g., 1-20 iterations, or fewer depending on processing hardware). Further, the regularization term may include an L2 norm penalty on parameter deltas relative to baseline parameters, and the bound may be enforced by rejecting updates that exceed a deviation threshold. Further, storing baseline model parameters in memory may include storing parameters in association with a model version identifier and storing a rollback copy such that updates remain reversible.

Further, in some embodiments, generating the feedback data may include generating a real-time control signal for a wearable device comprising at least one actuator. Further, the actuator may include at least one of a haptic motor, a piezoelectric actuator, a linear resonant actuator, an eccentric rotating mass motor, a speaker, or an electro-tactile output device. Further, the real-time control signal may be computed from (i) a deviation between the at least one actual metric and the at least one reference metric and (ii) a confidence score produced by the at least one AI model.

Further, in some embodiments, the confidence score may be generated from at least one of a model uncertainty estimate, a signal quality estimate derived from the noise characteristic, and/or a consistency measure across multiple sensors, and may be used to gate or scale the control signal to reduce false-positive cues. Further, transmitting the feedback data to the at least one user device may cause the wearable device to output a haptic cue during performance of the at least one physical activity to modify a biomechanical characteristic of the at least one physical activity, such as a joint angle, joint angular velocity, foot strike pattern, cadence, stride length, limb symmetry, trunk lean, or range of motion.

Further, in some embodiments, the real-time control signal may be generated subject to a latency constraint such that the haptic cue is output within a predetermined time window after receipt of a corresponding portion of the at least one content data. Further, the predetermined time window may be selected based on the physical activity type such that corrective feedback occurs within a time horizon that is perceptually and biomechanically relevant (e.g., within a fraction of a gait cycle or swing phase). Further, outputting the haptic cue within the predetermined time window may form a closed-loop corrective control of the biomechanical characteristic during the at least one physical activity.

Further, in some embodiments, the closed-loop control may include: receiving updated content data, recomputing the actual metric, recomputing deviation relative to the reference metric, and updating the control signal for a subsequent time window. Further, the system may log the deviation and the applied control cue to refine subsequent feedback and/or to provide post-session summaries.

Further, in some embodiments, the method may further include transmitting, to the at least one content source device, a capture-control command derived from the dynamically varied model parameters and the data characteristics to modify at least one capture setting comprising at least one of frame rate, exposure, gain, shutter speed, or sampling rate for subsequent content data, to reduce measurement error of the at least one actual metric relative to the at least one reference metric.

Further, in some embodiments, deriving the capture-control command may include mapping the data characteristics to a predicted measurement error contribution and selecting a capture setting update that reduces the predicted measurement error. For example, when compression artifact level exceeds a threshold, the capture-control command may increase bitrate or reduce compression; when motion blur is detected from content features, the capture-control command may decrease shutter time and adjust gain; and when sensor drift is detected from the noise characteristic, the capture-control command may increase sampling rate and/or trigger recalibration. Further, the capture-control command may be transmitted as a structured message including a device identifier, a setting identifier, and a setting value, thereby providing an objective, implementable interface.

Further, in some embodiments, the claimed operations improve computer-implemented biomechanical evaluation by (i) compensating for device-dependent variation, (ii) reducing measurement error and/or latency, and (iii) producing real-time corrective control outputs (e.g., haptics) and/or capture-control commands.

Further, in some embodiments, the “at least one reference data” may include reference motion data and/or reference biomechanical metrics for at least one physical activity, and may be sourced from at least one of a curated dataset, a clinician/coach-defined reference template, a personalized baseline for the user, or a population-level normative reference.

Further, in some embodiments, “feature representation” may include a vector, tensor, embedding, set of keypoints, skeleton sequence, or time-windowed latent representation produced by the AI model. Further, “characteristically aligned” may refer to the content feature representation and the reference feature representation satisfying a quantitative alignment criterion, such as the feature-discrepancy metric being below a threshold, being within a tolerance band, and/or converging within a bounded number of update iterations.

Further, in some embodiments, the disclosed operations are performed by a processing device executing instructions stored in non-transitory memory and by a communication device and storage device, as recited in the claims. Further, the disclosure describes specific algorithmic steps (serialization, encoding, schema validation, quantization selection, jitter buffering, etc.). Further, the disclosure avoids purely functional “means for” claiming by tying the functions to specific computing structures (processor, memory, tokenizer/encoder, transformer inference engine, schema validator, network measurement routines, jitter buffer routines).

Further, the dynamic user characteristic value is treated as input data, and the instruction is a directive that uses that value to set constraints. The instruction may specify constraints computed from the dynamic user characteristic (e.g., a fatigue value causes a reduction of feedback intensity).

Further, in some embodiments, a “time-window identifier” may refer to a value identifying a temporal segment of the activity. Further, the time-window identifier may include a time interval [t_start, t_end], a frame index range, a sample index range, an epoch timestamp bucket, or a repetition number and sub-phase identifier.

Further, in some embodiments, a “device characteristic” may refer to a value describing a capability, limitation, operation, or state of the user device. Further, the device characteristic may include available memory, processor type (e.g., CPU/GPU/NPU), current CPU/GPU load, battery level, thermal state, network type, measured bandwidth, measured latency, packet loss rate, display resolution, frame rate, availability of haptics, availability of audio output, or sensor availability state.

Further, in some embodiments, a “dynamic user characteristic” may refer to a user-associated value that changes over time or over sessions. Further, the dynamic user characteristic may include a fatigue indicator, a responsiveness metric to prior feedback, a range-of-motion constraint observed during the activity, a movement variability metric, a tremor metric, a discomfort indicator provided by the user, a safety risk score computed from deviation patterns, or a confidence score associated with the spatial characteristic data.

Further, in some embodiments, an “instruction” may refer to a machine-executable or machine-interpretable directive that constrains how the large language model generates outputs. Further, the instruction may specify an output format (e.g., schema), a target modality, a safety constraint, a timing constraint, a maximum payload size, or a target end-to-end latency. Further, an instruction is distinct from an “input” in that the instruction governs generation behavior, while the input supplies data used to generate a result.

Further, in some embodiments, an “input” may refer to the data provided to the large language model for inference, including the content data and/or spatial characteristic data, the time-window identifier, the user query, and device characteristics.

Further, in some embodiments, a “machine-readable spatio-temporal feedback instruction set” may refer to a structured data object that is parseable and executable by software on the user device. Further, the machine-readable spatio-temporal feedback instruction set may be represented as a JSON object, a protocol buffer (protobuf) message, a CBOR payload, an XML message, or another structured format, and may include time-indexed control primitives.

Further, in some embodiments, a “control primitive” may refer to a structured instruction specifying a feedback action at a time or time offset, including a corrective rotation vector, corrective translation vector, pose target, haptic event, audio event, or visual overlay event.

Further, in some embodiments, the “at least one content source device” is and/or may include at least one of a camera device, a smartphone, a wearable device, an IMU-based sensor module, a depth sensor, a pressure-sensing insole, a heart-rate sensor, an EMG sensor, or a motion-capture device. Further, the “at least one user device” is and/or includes at least one of a smartphone, tablet, head-mounted display, smartwatch, wearable device, or computer.

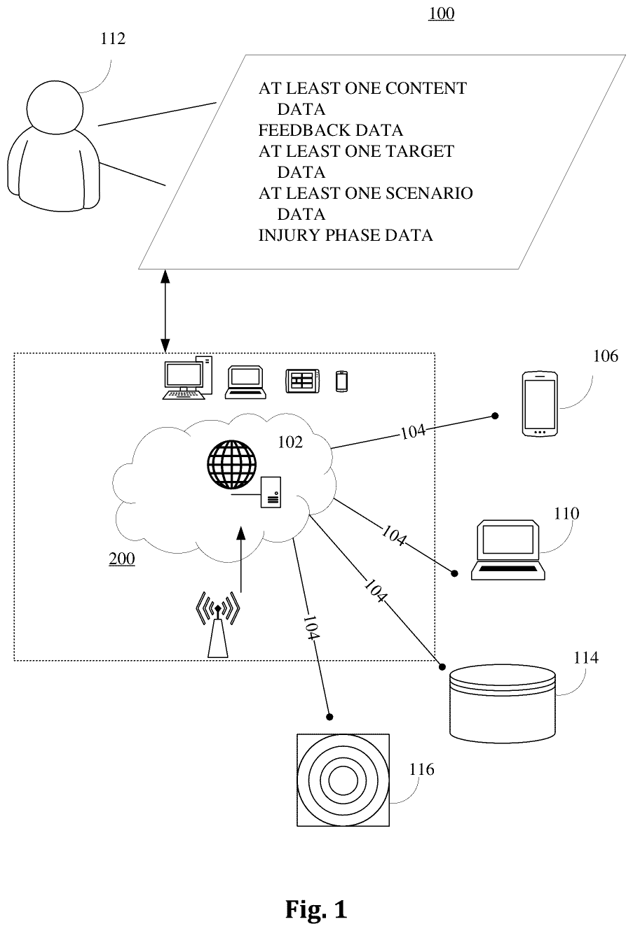

FIG. 1 is an illustration of an online platform 100 consistent with various embodiments of the present disclosure. By way of non-limiting example, the online platform 100 may be hosted on a centralized server 102, such as, for example, a cloud computing service. The centralized server 102 may communicate with other network entities, such as, for example, a mobile device 106 (such as a smartphone, a laptop, a tablet computer etc.), other electronic devices 110 (such as desktop computers, server computers etc.), databases 114, and sensors 116 over a communication network 104, such as, but not limited to, the Internet. Further, users of the online platform 100 may include relevant parties such as, but not limited to, end-users, administrators, service providers, service consumers, and so on. Accordingly, in some instances, electronic devices operated by the one or more relevant parties may be in communication with the platform.

A user 112, such as the one or more relevant parties, may access online platform 100 through a web based software application or browser. The web based software application may be embodied as, for example, but not limited to, a website, a web application, a desktop application, and a mobile application compatible with a computing device 200.

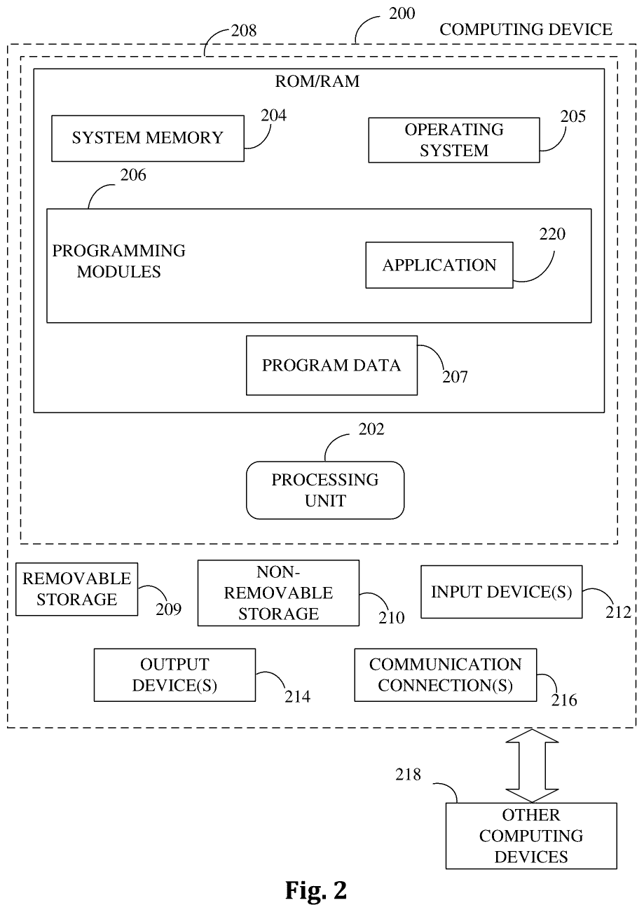

With reference to FIG. 2, a system consistent with an embodiment of the disclosure may include a computing device or cloud service, such as computing device 200. In a basic configuration, computing device 200 may include at least one processing unit 202 and a system memory 204. Depending on the configuration and type of computing device, system memory 204 may comprise, but is not limited to, volatile (e.g. random-access memory (RAM)), non-volatile (e.g. read-only memory (ROM)), flash memory, or any combination. System memory 204 may include operating system 205, one or more programming modules 206, and may include a program data 207. Operating system 205, for example, may be suitable for controlling computing device 200's operation. In one embodiment, programming modules 206 may include image-processing module, machine learning module. Furthermore, embodiments of the disclosure may be practiced in conjunction with a graphics library, other operating systems, or any other application program and are not limited to any particular application or system. This basic configuration is illustrated in FIG. 2 by those components within a dashed line 208.

Computing device 200 may have additional features or functionality. For example, computing device 200 may also include additional data storage devices (removable and/or non-removable) such as, for example, magnetic disks, optical disks, or tape. Such additional storage is illustrated in FIG. 2 by a removable storage 209 and a non-removable storage 210. Computer storage media may include volatile and non-volatile, removable and non-removable media implemented in any method or technology for storage of information, such as computer-readable instructions, data structures, program modules, or other data. System memory 204, removable storage 209, and non-removable storage 210 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 200. Any such computer storage media may be part of device 200. Computing device 200 may also have input device(s) 212 such as a keyboard, a mouse, a pen, a sound input device, a touch input device, a location sensor, a camera, a biometric sensor, etc. Output device(s) 214 such as a display, speakers, a printer, etc. may also be included. The aforementioned devices are examples and others may be used.

Computing device 200 may also contain a communication connection 216 that may allow device 200 to communicate with other computing devices 218, such as over a network in a distributed computing environment, for example, an intranet or the Internet. Communication connection 216 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 204, including operating system 205. While executing on processing unit 202, programming modules 206 (e.g., application 220 such as a media player) may perform processes including, for example, one or more stages of methods, algorithms, systems, applications, servers, databases as described above. The aforementioned process is an example, and processing unit 202 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

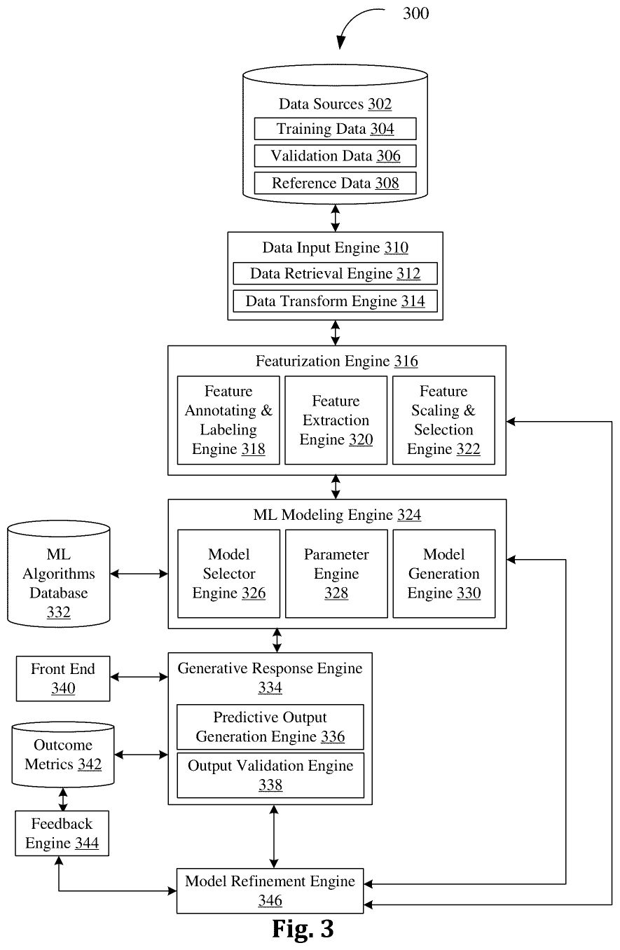

FIG. 3 is a block diagram illustrating a machine-learning system 300 for implementing various embodiments of this disclosure, in accordance with some embodiments. Although the disclosed machine-learning system 300 depicts particular system components and an arrangement of such components, the given depiction is to facilitate a discussion of the present technology and should not be considered limiting unless specified in the appended claims. For, example some components that are illustrated as separate, may be combined with other components and some components may be divided into separate components.

Accordingly, the machine-learning system 300 may include a plurality of interrelated modules and engines configured to implement a machine-learning pipeline. Further, the machine-learning system 300 may include a data sources module 302 that is made up of a training data repository 304, a validation data repository 306, and a reference data repository 308, each repository being configured to store respective classes of input records and reference information. Further, the machine-learning system 300 may include a data input engine 310 configured to receive data from the data sources module 302. Further, the data input engine 310 may include a data retrieval engine 312 configured to access and ingest data from the repositories (304, 306, 308), and a data transform engine 314 configured to perform initial normalization, parsing and format conversion on the ingested data. Further, the machine-learning system 300 may include a featurization engine 316 configured to prepare temporal and predictive representations of transformed data. Further, the featurization engine 316 may include a feature annotating & labeling engine 318 for applying labels and annotations to data instances, a feature extraction engine 320 for deriving feature vectors and candidate predictors, and a feature scaling & selection engine 322 for performing numerical scaling, dimensionality reduction and selection of salient features. Further, the machine-learning system 300 may include a machine learning (ML) modeling engine 324 configured to construct predictive models from selected features. Further, the ML modeling engine 324 may include a model selector engine 326 for selecting among candidate model classes, a parameter engine 328 for determining and tuning hyper parameters, and a model generation engine 330 for instantiating and training model artifacts according to selected architectures and parameters. Further, the machine-learning system 300 may include an ML algorithms database 332 configured to store algorithmic implementations, model templates and associated metadata and to be accessible by components of the ML modeling engine 324. Further, the machine-learning system 300 may include a generative response engine 334 configured to produce user-facing outputs based on the trained models. Further, the generative response engine 334 may include a predictive output generation engine 336 for generating predictions or synthesized responses and an output validation engine 338 for verifying, filtering and validating generated outputs against predefined criteria and reference data. Further, the machine-learning system 300 may include a front end 340 configured to present validated outputs to end users and to collect interaction signals. Further, the machine-learning system 300 may include an outcome metrics module 342 configured to compute performance measures, accuracy statistics and other evaluation metrics derived from model outputs and user interactions. Further, the machine-learning system 300 may include a feedback engine 344 configured to aggregate outcome metrics and user feedback and to format such information for reuse. Further, the machine-learning system 300 may include a model refinement engine 346 configured to receive feedback from the feedback engine 344 and the outcome metrics module 342, and to effect iterative updates to the ML modeling engine 324 and to the ML algorithms database 332. Further, the components are communicatively coupled so that data and control signals are exchanged among the repositories (304, 306, 308), the data input engine 310, the featurization engine 316, the ML modeling engine 324 (with algorithmic support from the ML algorithms database 332), the generative response engine 334 and the front end 340 for output generation. Further, the outcome metrics 342 and feedback engine 344 provide closed-loop signals to the model refinement engine 346 to enable retraining, parameter adjustment and algorithm selection, thereby enabling cooperative execution of data acquisition, feature engineering, model construction, output generation, validation, evaluation and iterative refinement within the disclosed machine-learning system 300.

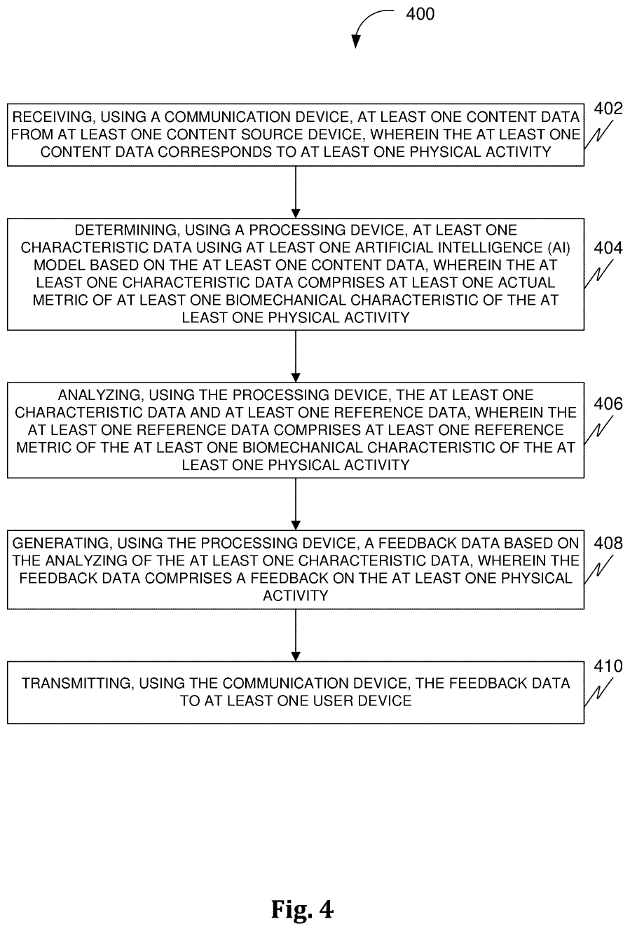

FIG. 4 illustrates a flowchart of a method 400 of facilitating performance evaluation of physical activities, in accordance with some embodiments. Further, the method 400 may include a step 402 of receiving, using a communication device 802, one or more content data from one or more content source devices 806. Further, the one or more content data corresponds to one or more physical activities. Further, the method 400 may include a step 404 of determining, using a processing device 804, one or more characteristic data using one or more Artificial Intelligence (AI) models based on the one or more content data. Further, the one or more characteristic data includes one or more actual metrics of one or more biomechanical characteristics of the one or more physical activities. Further, the one or more biomechanical characteristics of the one or more physical activities are and/or the one or more biomechanical characteristics of one or more users performing the one or more physical activities. Further, the one or more users may include individuals, athletes, etc. Further, the method 400 may include a step 406 of analyzing, using the processing device 804, the one or more characteristic data and one or more reference data. Further, the one or more reference data includes one or more reference metrics of the one or more biomechanical characteristics of the one or more physical activities. Further, the method 400 may include a step 408 of generating, using the processing device 804, a feedback data based on the analyzing of the one or more characteristic data. Further, the feedback data includes a feedback on the one or more physical activities. Further, the method 400 may include a step 410 of transmitting, using the communication device 802, the feedback data to one or more user devices 808.

In some embodiments, the one or more physical activities include two or more phases. Further, the one or more AI models may be configured to evaluate the one or more biomechanical characteristics of the one or more physical activities comprising the two or more phases. Further, the method 400 further includes analyzing, using the processing device 804, the one or more content data using the one or more AI models. Further, the determining of the one or more characteristic data may be further based on the analyzing of the one or more content data using the one or more AI models.

In some embodiments, the method 400 may further include determining, using the processing device 804, a deviation of the one or more actual metrics from the one or more reference metrics based on the analyzing of the one or more characteristic data and the one or more reference data. Further, the generating of the feedback data may be further based on the determining of the deviation.

In some embodiments, the generating of the feedback data includes generating one or more scores using the one or more AI models based on the determining of the deviation. Further, the one or more AI models may be configured to generate the one or more scores for the deviation based on a Gaussian distribution associated with the one or more reference metrics. Further, the feedback data includes the one or more scores.

In some embodiments, the method 400 may further include retrieving, using a storage device 902, the one or more reference data from a database based on the one or more content data. Further, the database includes two or more reference data corresponding to two or more physical activities. Further, the analyzing of the one or more characteristic data may be further based on the retrieving of the one or more reference data.

In some embodiments, the one or more reference data includes one or more first feedback data corresponding to one or more first content data. Further, the one or more first content data corresponds to one or more first physical activities. Further, the one or more first content data may be received at a first time instance. Further, the one or more content data may be received at a second time instance. Further, the second time instance occurs after the first time instance. Further, the one or more reference metrics include one or more first actual metrics corresponding to the one or more biomechanical characteristics of the one or more first physical activities.

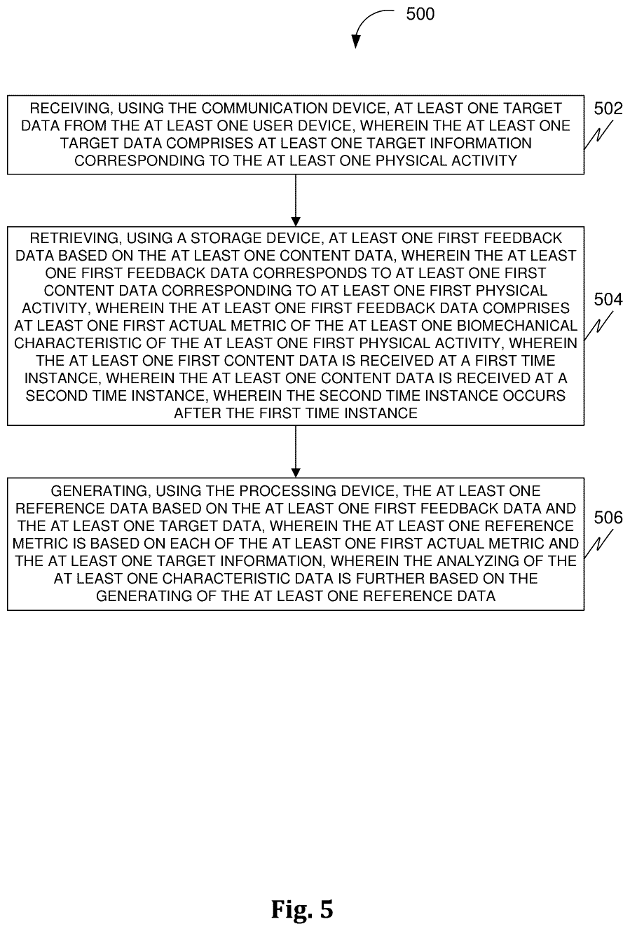

FIG. 5 illustrates a flowchart of a method 500 of facilitating performance evaluation of physical activities including generating, using the processing device 804, the at least one reference data, in accordance with some embodiments. Further, in some embodiments, the method 500 further may include a step 502 of receiving, using the communication device 802, one or more target data from the one or more user devices 808. Further, the one or more target data includes one or more target information corresponding to the one or more physical activities. Further, in some embodiments, the method 500 further may include a step 504 of retrieving, using a storage device 902, one or more first feedback data based on the one or more content data. Further, the one or more first feedback data corresponds to one or more first content data corresponding to one or more first physical activities. Further, the one or more first feedback data includes one or more first actual metrics of the one or more biomechanical characteristics of the one or more first physical activities. Further, the one or more first content data may be received at a first time instance. Further, the one or more content data may be received at a second time instance. Further, the second time instance occurs after the first time instance. Further, in some embodiments, the method 500 further may include a step 506 of generating, using the processing device 804, the one or more reference data based on the one or more first feedback data and the one or more target data. Further, the one or more reference metrics may be based on each of the one or more first actual metrics and the one or more target information. Further, the analyzing of the one or more characteristic data may be further based on the generating of the one or more reference data.

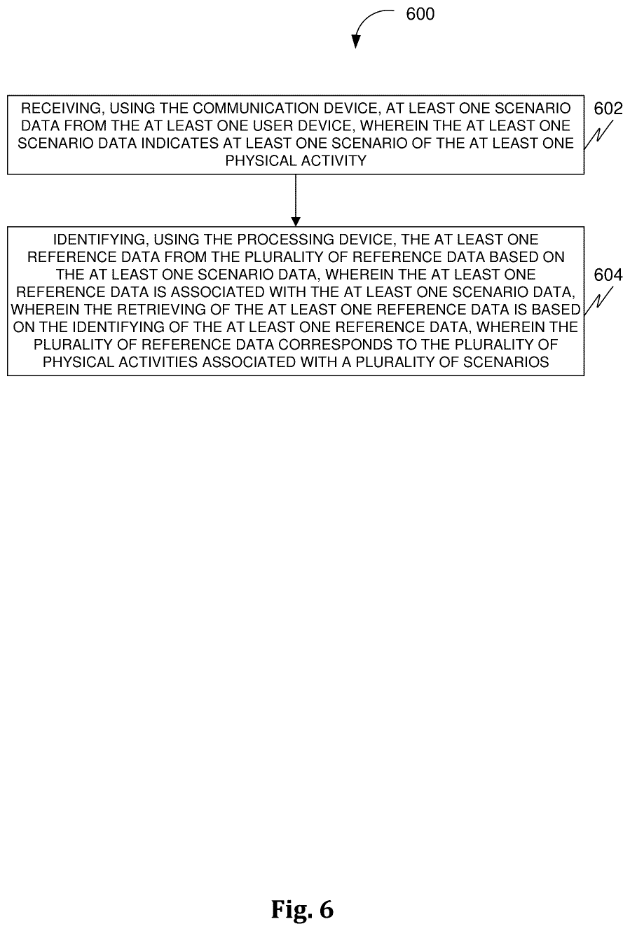

FIG. 6 illustrates a flowchart of a method 600 of facilitating performance evaluation of physical activities including identifying, using the processing device 804, the at least one reference data from the plurality of reference data, in accordance with some embodiments. Further, in some embodiments, the method 600 further may include a step 602 of receiving, using the communication device 802, one or more scenario data from the one or more user devices 808. Further, the one or more scenario data indicate one or more scenarios of the one or more physical activities. Further, in some embodiments, the method 600 further may include a step 604 of identifying, using the processing device 804, the one or more reference data from the two or more reference data based on the one or more scenario data. Further, the one or more reference data may be associated with the one or more scenario data. Further, the retrieving of the one or more reference data may be based on the identifying of the one or more reference data. Further, the two or more reference data correspond to the two or more physical activities associated with two or more scenarios.

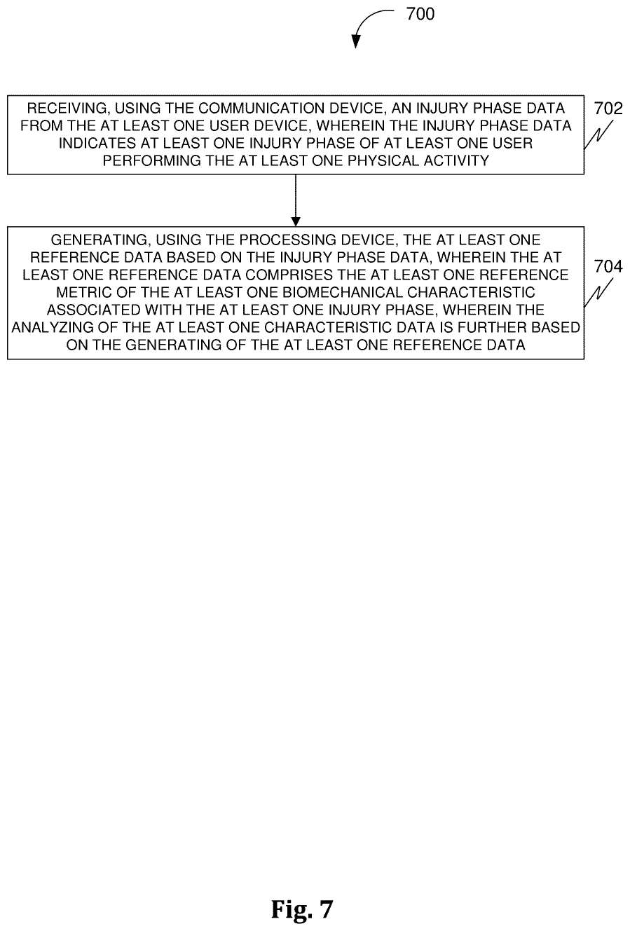

FIG. 7 illustrates a flowchart of a method 700 of facilitating performance evaluation of physical activities including generating, using the processing device 804, the at least one reference data, in accordance with some embodiments. Further, in some embodiments, the method 700 further may include a step 702 of receiving, using the communication device 802, an injury phase data from the one or more user devices 808. Further, the injury phase data indicates one or more injury phases of one or more users performing the one or more physical activities. Further, in some embodiments, the method 700 further may include a step 704 of generating, using the processing device 804, the one or more reference data based on the injury phase data. Further, the one or more reference data includes the one or more reference metrics of the one or more biomechanical characteristics associated with the one or more injury phases. Further, the analyzing of the one or more characteristic data may be further based on the generating of the one or more reference data.

In some embodiments, the method 400 may further include generating, using the processing device 804, one or more spatial data based on the one or more content data. Further, the one or more spatial data includes one or more three-dimensional metrics corresponding to the one or more physical activities. Further, the determining of the one or more characteristic data may be further based on the one or more spatial data. Further, the one or more AI models may be configured to determine the one or more biomechanical characteristics of the one or more physical activities based on the one or more spatial data.

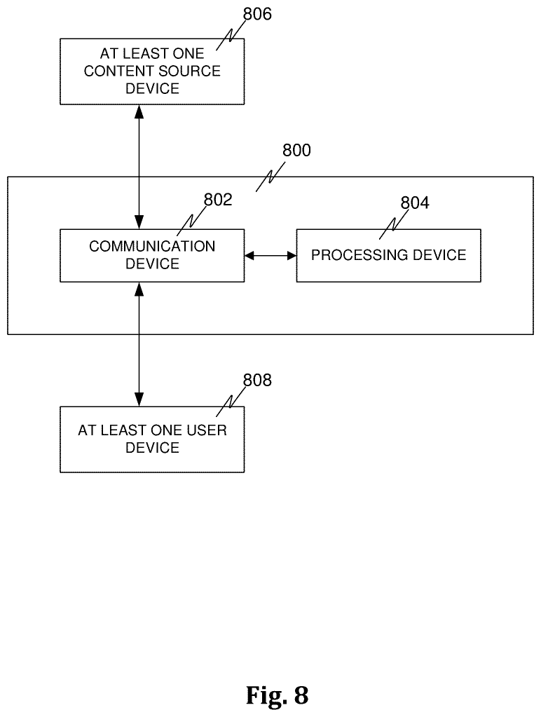

FIG. 8 illustrates a block diagram of a system 800 of facilitating performance evaluation of physical activities, in accordance with some embodiments. Further, the system 800 may include a communication device 802. Further, the communication device 802 may be configured for receiving one or more content data from one or more content source devices 806. Further, the one or more content data corresponds to one or more physical activities. Further, the communication device 802 may be configured for transmitting a feedback data to one or more user devices 808. Further, the system 800 may include a processing device 804 communicatively coupled with the communication device 802. Further, the processing device 804 may be configured for determining one or more characteristic data using one or more Artificial Intelligence (AI) models based on the one or more content data. Further, the one or more characteristic data includes one or more actual metrics of one or more biomechanical characteristics of the one or more physical activities. Further, the processing device 804 may be configured for analyzing the one or more characteristic data and one or more reference data. Further, the one or more reference data includes one or more reference metrics of the one or more biomechanical characteristics of the one or more physical activities. Further, the processing device 804 may be configured for generating the feedback data based on the analyzing of the one or more characteristic data. Further, the feedback data includes a feedback on the one or more physical activities.

In some embodiments, the one or more physical activities include two or more phases. Further, the one or more AI models may be configured to evaluate the one or more biomechanical characteristics of the one or more physical activities comprising the two or more phases. Further, the processing device 804 may be further configured for analyzing the one or more content data using the one or more AI models. Further, the determining of the one or more characteristic data may be further based on the analyzing of the one or more content data using the one or more AI models.

In some embodiments, the processing device 804 may be further configured for determining a deviation of the one or more actual metrics from the one or more reference metrics based on the analyzing of the one or more characteristic data and the one or more reference data. Further, the generating of the feedback data may be further based on the determining of the deviation.

In some embodiments, the generating of the feedback data includes generating one or more scores using the one or more AI models based on the determining of the deviation. Further, the one or more AI models may be configured to generate the one or more scores for the deviation based on a Gaussian distribution associated with the one or more reference metrics. Further, the feedback data includes the one or more scores.

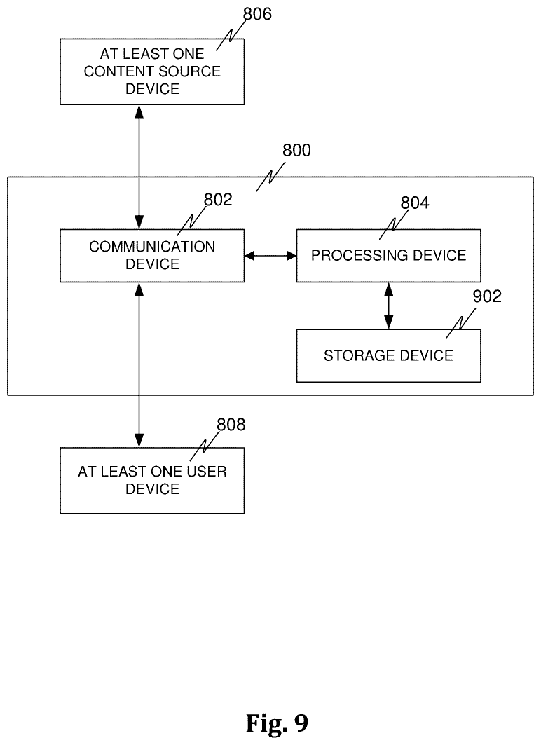

FIG. 9 illustrates a block diagram of the system 800 of facilitating performance evaluation of physical activities, in accordance with some embodiments. In some embodiments, the system 800 may further include a storage device 902 communicatively coupled with the communication device 802. Further, the storage device 902 may be configured for retrieving the one or more reference data from a database based on the one or more content data. Further, the database includes two or more reference data corresponding to two or more physical activities. Further, the analyzing of the one or more characteristic data may be further based on the retrieving of the one or more reference data.

In some embodiments, the one or more reference data includes one or more first feedback data corresponding to one or more first content data. Further, the one or more first content data corresponds to one or more first physical activities. Further, the one or more first content data may be received at a first time instance. Further, the one or more content data may be received at a second time instance. Further, the second time instance occurs after the first time instance. Further, the one or more reference metrics include one or more first actual metrics corresponding to the one or more biomechanical characteristics of the one or more first physical activities.

In some embodiments, the communication device 802 may be further configured for receiving one or more target data from the one or more user devices 808. Further, the one or more target data includes one or more target information corresponding to the one or more physical activities. Further, the system 800 further includes a storage device 902 communicatively coupled with the communication device 802. Further, the storage device 902 may be configured for retrieving one or more first feedback data based on the one or more content data. Further, the one or more first feedback data corresponds to one or more first content data of the one or more first physical activities. Further, the one or more first feedback data includes one or more first actual metric corresponding to the one or more biomechanical characteristics of the one or more first physical activities. Further, the one or more first content data may be received at a first time instance. Further, the one or more content data may be received at a second time instance. Further, the second time instance occurs after the first time instance. Further, the processing device 804 may be further communicatively coupled with the storage device 902. Further, the processing device 804 may be configured for generating the one or more reference data based on the one or more first feedback data and the one or more target data. Further, the one or more reference metrics may be based on each of the one or more first actual metrics and the one or more target information. Further, the analyzing of the one or more characteristic data may be further based on the generating of the one or more reference data.

In some embodiments, the communication device 802 may be further configured for receiving one or more scenario data from the one or more user devices 808. Further, the one or more scenario data indicate one or more scenarios of the one or more physical activities. Further, the processing device 804 may be configured for identifying the one or more reference data form the two or more reference data based on the one or more scenario data. Further, the one or more reference data may be associated with the one or more scenario data. Further, the retrieving of the one or more reference data may be based on the identifying of the one or more reference data. Further, the two or more reference data corresponds to the two or more physical activities associated with two or more scenarios.

In some embodiments, the communication device 802 may be further configured for receiving an injury phase data from the one or more user devices 808. Further, the injury phase data indicates one or more injury phases of one or more users performing the one or more physical activities. Further, the processing device 804 may be further configured for generating the one or more reference data based on the injury phase data. Further, the one or more reference data includes the one or more reference metrics of the one or more biomechanical characteristics associated with the one or more injury phases. Further, the analyzing of the one or more characteristic data may be further based on the generating of the one or more reference data.

In some embodiments, the processing device 804 may be further configured for generating one or more spatial data based on the one or more content data. Further, the one or more spatial data includes one or more three-dimensional metrics corresponding to the one or more physical activities. Further, the determining of the one or more characteristic data may be further based on the one or more spatial data. Further, the one or more AI models may be configured to determine the one or more biomechanical characteristics of the one or more physical activities based on the one or more spatial data.

In some embodiments, the one or more user devices may be associated with one or more experts of the one or more physical activities. Further, the one or more target data comprises an exemplar data comprising a standard reference of the one or more biomechanical characteristics of the one or more physical activities.

In some embodiments, the one or more first feedback data may correspond to two or more players characterized by one or more common characteristics.

In some embodiments, the one or more common characteristic comprises one or more of an age, a skill, and a position.

In some embodiments, the one or more first feedback data may correspond to a first player. Further, the first player may be characterized by a first characteristic. Further, the one or more physical activities may be performed by a second player. Further, the second player may be characterized by a second characteristic. Further, the first characteristic and the second characteristic may be same.

In some embodiments, the one or more first feedback data may correspond to a first player. Further, the one or more physical activities may be performed by the first player. Further, the one or more first feedback data comprises a historical performance data of the first player.

In some embodiments, the method 400 may further include determining, using the processing device 804, a sport type of the one or more physical activities based on the one or more content data. Further, the method 400 may further include identifying, using the processing device 804, the one or more reference data from the two or more reference data based on the sport type. Further, the retrieving of the one or more reference data may be further based on the identifying of the one or more reference data from the two or more reference data based on the sport type. Further, the two or more reference data may be associated with two or more sport types. Further, the one or more reference data may be associated with the sport type of the one or more content data.

In some embodiments, the method 400 may further include determining, using the processing device 804, an activity type of the one or more physical activities based on the one or more content data. Further, the method 400 may further include identifying, using the processing device 804, the one or more reference data from the two or more reference data based on the activity type. Further, the retrieving of the one or more reference data may be further based on the identifying of the one or more reference data from the two or more reference data based on the activity type. Further, the two or more reference data may be associated with two or more activity types. Further, the one or more reference data may be associated with the activity type of the one or more content data.

In some embodiments, the one or more physical activities may be performed by one or more athletes. Further, the method 400 may further include identifying, using the processing device, one or more athlete data from two or more athlete data based on the one or more content data. Further, the one or more athlete data comprises one or more athlete information of the one or more athletes. Further, the two or more athlete data comprises two or more athlete information of two or more athletes. Further, the retrieving of the one or more reference data may be further based on the identifying of the one or more athlete data from the two or more athlete data based on the one or more content data.

In some embodiments, the one or more athlete information comprises one or more of one or more athlete profiles of the one or more athletes.

In some embodiments, the one or more athlete information indicates one or more skill levels of the one or more athletes.

In some embodiments, the one or more physical activities may be performed by one or more users. Further, the method 400 may further include determining, using the processing device 804, one or more positions of the one or more content source devices in relation to the one or more users based on the one or more content data. Further, the method 400 may further include identifying, using the processing device 804, the one or more reference data from the two or more reference data based on the one or more positions. Further, the retrieving of the one or more reference data may be further based on the identifying of the one or more reference data from the two or more reference data based on the one or more positions. Further, the two or more reference data may be associated with two or more positions of the one or more content source devices in relation to the one or more users. Further, the one or more reference data may be associated with the one or more positions of the one or more content data.

In some embodiments, the one or more content source devices comprise one or more cameras. Further, the one or more positions comprise one or more camera positions.

In some embodiments, the method 400 may further include receiving, using the communication device 802, one or more training objectives from the one or more user devices. Further, the retrieving of the one or more reference data is further based on the one or more training objectives. Further, the one or more reference data is associated with the one or more training objectives.

In some embodiments, the one or more physical activities may be performed by a user. Further, the method 400 may further include retrieving, using the storage device 902, two or more feedback data of the user based on the one or more content data. Further, the two or more feedback data indicate two or more performance feedback of the user. Further, the method 400 may further include updating, using the processing device, one or more first reference data based on the two or more feedback data of the user to obtain the one or more reference data. Further, the analyzing of the one or more characteristic data is based on the updating of the one or more first reference data.

In some embodiments, the two or more feedback data correspond to two or more physical activities performed by the user over a time interval.

In some embodiments, the determining of the one or more characteristic data comprises determining two or more actual metrics of the one or more physical activities. Further, the generating of the feedback data comprises generating the one or more scores using the one or more AI models based on the two or more actual metrics of the one or more physical activities. Further, the one or more AI models may be configured to generate the one or more scores based on a weighted combination of the two or more actual metrics of the one or more physical activities.

In some embodiments, the method 400 may include assigning, using the processing device 804, two or more weights to the two or more actual metrics using the one or more AI models based on the determining of the two or more actual metrics. Further, the one or more AI models may be configured to assign the two or more weights to the two or more actual metrics based on the one or more of a coaching objective, an injury risk preference, and an athlete profile. Further, the generating of the one or more scores may be further based on the assigning of the two or more weights.

In some embodiments, the method 400 may include processing, using the processing device 804, the one or more characteristic data comprising the two or more actual metrics. Further, the method 400 may include obtaining, using the processing device 804, one or more processed characteristic data based on the processing of the one or more characteristic data. Further, the analyzing of the one or more characteristic data may be further based on the obtaining of the one or more processed characteristic data.

In some embodiments, the processing of the one or more characteristic data comprises normalizing the two or more actual metrics. Further, the obtaining of the one or more processed characteristic data is based on the normalizing of the two or more actual metrics. Further, the one or more processed characteristic data comprises two or more normalized actual metrics of the one or more physical activities.

In some embodiments, the normalizing of the two or more actual metrics comprises normalizing the two or more actual metrics based on one or more normalizing factors. Further, the one or more normalizing factors comprise one or more of a body dimension, a height, a limb length, and a camera geometry.

In some embodiments, the method 400 may further include aggregating, using the processing device 804, the one or more processed characteristic data based on the obtaining of the one or more processed characteristic data. Further, the method 400 may further include obtaining, using the processing device 804, an aggregated characteristic data based on the aggregating the one or more processed characteristic data. Further, the analyzing of the one or more characteristic data and the one or more reference data may be based on the obtaining of the aggregated characteristic data. Further, the analyzing of the one or more characteristic data and the one or more reference data comprises analyzing the aggregated characteristic data and the one or more reference data.

In some embodiments, the method 400 may further include segmenting, using the processing device 804, the one or more content data into two or more content data. Further, the two or more content data correspond to the two or more phases of the one or more physical activities. Further, the analyzing of the one or more content data may be based on the segmenting of the one or more content data. Further, the analyzing of the one or more content data includes analyzing one or more of the two or more content data using the one or more AI models.

In some embodiments, the one or more target information includes one or more target metrics of the one or more biomechanical characteristics of the one or more physical activities. Further, the one or more target metrics may be specified by one or more coaches.

In some embodiments, the one or more physical activities may be performed by one or more users. Further, the one or more user devices 808 may be associated with one or more users.

In some embodiments, the one or more user devices 808 may be associated with one or more users. Further, the one or more physical activities may be performed by the one or more users.

In some embodiments, the one or more physical activities correspond to one or more sports. Further, the one or more scenarios may be associated with the one or more sports. Further, the one or more physical activities may be performed based on the one or more scenarios.

In some embodiments, the one or more sports include a basketball sport. Further, the one or more scenarios include one or more of a guarded shot scenario and an unguarded shot scenario.

In some embodiments, the method 700 may further include retrieving, using a storage device 902, one or more first feedback data based on the one or more content data. Further, the one or more first feedback data corresponds to one or more first content data corresponding to one or more first physical activities. Further, the one or more first feedback data includes one or more first actual metrics corresponding to the one or more biomechanical characteristics of the one or more first physical activities. Further, the one or more first content data may be received at a first time instance. Further, the one or more content data may be received at a second time instance. Further, the second time instance occurs after the first time instance. Further, the generating of the one or more reference data may be further based on the retrieving of the one or more first feedback data. Further, the generating of the one or more reference data includes modifying the one or more first actual metric information based on the one or more injury phases to obtain the one or more reference metrics.

In some embodiments, the one or more injury phases include one or more of a pre-injury phase and an injury-recovery phase.

In some embodiments, the one or more injury phases include a rehabilitation phase.

In some embodiments, the one or more physical activities correspond to a basketball shot. Further, the two or more phases include a pocket phase, a release phase, and a post-release phase.

In some embodiments, the one or more characteristic data comprises two or more actual metrics comprising two or more subsets of metrics. Further, the two or more subsets of metrics correspond to the two or more phases of the one or more physical activities.

In some embodiments, the two or more phases comprise a first phase and a second phase. Further, the two or more subsets of metrics comprise a first subset of metrics corresponding to the first phase and a second subset of metrics corresponding to the second phase. Further, the analyzing of the one or more characteristic data and the one or more reference data comprises analyzing the first subset of metrics and the one or more reference data for the first phase of the one or more physical activities. Further, the analyzing of the one or more characteristic data and the one or more reference data comprises analyzing the second subset of metrics and the one or more reference data for the second phase of the one or more physical activities. Further, the generating of the feedback data is based on each of the analyzing of the first subset of metrics and the one or more reference data, and the analyzing of the second subset of metrics and the one or more reference data.

In some embodiments, the generating of the feedback data comprises generating a first score based on the analyzing of the first subset of metrics and the one or more reference data. Further, the first score corresponds to the first phase of the one or more physical activities. Further, the generating of the feedback data comprises generating a second score based on the analyzing of the second subset of metrics and the one or more reference data. Further, the second score corresponds to the second phase of the one or more physical activities. Further, the generating of the feedback data comprises generating the one or more scores of the one or more physical activities based on each of the first score and the second score.

In some embodiments, the one or more physical activities may be performed by one or more users. Further, the one or more biomechanical characteristics of the one or more physical activities correspond to one or more of a joint angle of the one or more users, a body alignment of the one or more users, and a movement trajectory of the one or more users.

In some embodiments, the one or more spatial data include a quaternion-based three-dimensional spatial data. Further, the quaternion-based three-dimensional spatial data includes a quaternion representing the one or more physical activities.

In some embodiments, the one or more physical activities may be performed by one or more users using one or more objects. Further, the quaternion represents a rotational orientation of one or more of a body part of the one or more users and the one or more objects.

In some embodiments, the one or more spatial data comprises the quaternion representing one or more orientations of one or more of one or more body parts of a user and the one or more objects associated with the one or more physical activities. Further, the one or more body parts comprise a first body part of the user and the second body part of the user. Further, the determining of the one or more characteristic data based on the one or more spatial data comprises determining a relative orientation metric based on the quaternion representing the one or more orientations of the first body part and the second body part. Further, the one or more actual metrics comprise the relative orientation metric. Further, the relative orientation metric quantifies an orientation of the first body part in relation to the second body part.

In some embodiments, the one or more actual metrics comprise one or more rotational metrics corresponding to the rotational orientation.

In some embodiments, the one or more rotational metrics comprises one or more of an angular velocity, an angular acceleration, a rotational stability index, and a joint rotational range.

In some embodiments, the quaternion represents the rotational orientation of one or more of the body part and the one or more objects using one or more of a vector component and a scalar component.

In some embodiments, the quaternion represents an axis of the rotational orientation using the vector component.

In some embodiments, the quaternion represents one or more of a magnitude of the rotational orientation and an angle of the rotational orientation using the scalar component.

In some embodiments, the one or more AI models comprise one or more probabilistic models. Further, the one or more probabilistic models may be configured to generate the one or more scores based on the Gaussian distribution.

In some embodiments, the one or more probabilistic models may be personalized to one or more athletes using one or more historical data of the one or more athletes. Further, the one or more physical activities may be performed by the one or more athletes.

In some embodiments, the one or more AI models comprise one or more mixture models. Further, the one or more mixture models may be configured to perform one or more statistical operations to generate the one or more scores.

In some embodiments, the method 400 may include generating, using the processing device 804, one or more confidence scores of the one or more characteristic data based on the determining of the one or more characteristic data. Further, the one or more confidence scores quantify a reliability of the one or more actual metrics of the one or more physical activities. Further, the method 400 may include analyzing, using the processing device 804, the one or more confidence scores based on a confidence score threshold. Further, the method 400 may include determining, using the processing device 804, a result based on the analyzing of the one or more confidence scores based on the confidence score threshold. Further, the generating of the feedback data may be further based on the determining of the result.

In some embodiments, the determining of the result comprises one of determining a positive result and determining a negative result based on the analyzing of the one or more confidence scores based on the confidence score threshold. Further, the generating of the feedback data may be based on the determining of the positive result.

In some embodiments, the positive result may indicate an exceedance of the one or more confidence scores in relation to the confidence score threshold. Further, the negative result may indicate a non-exceedance of the one or more confidence scores in relation to the confidence score threshold.

In some embodiments, the one or more content data comprises one or more noise data. Further, the method 400 may include processing, using the processing device 804, the one or more content data. Further, the method 400 may include obtaining, using the processing device 804, one or more processed content data. Further, the one or more processed content data lacks the one or more noise data.

In some embodiments, the processing of the one or more content data comprises one or more of smoothing the one or more content data, filtering the one or more content data, and performing an imputation on the one or more content data.

In some embodiments, the one or more noise data comprises one or more of a missing content data and a noisy content data.

In some embodiments, the one or more physical activities may be performed by one or more users. Further, the feedback provides coaching to the one or more users.

In some embodiments, the feedback data instructs the one or more users to correct one or more mechanics of the one or more physical activities.

In some embodiments, the one or more users include one or more athletes.

In some embodiments, the one or more content data includes one or more video data.

In some embodiments, the one or more content data includes one or more sensor data.

In some embodiments, the one or more content source devices 806 is and/or include one or more cameras.

In some embodiments, the one or more content source devices 806 include and/or is one or more sensors. Further, the one or more sensors include and/or is heterogeneous sensors.

In some embodiments, the one or more physical activities may be performed by the one or more users using one or more objects. Further, the one or more AI models may be configured to evaluate the one or more biomechanical characteristics of the one or more users in relation to the one or more objects. Further, the one or more AI models may be further configured to evaluate one or more characteristics of the one or more objects.

In some embodiments, the one or more physical activities include a sport activity. Further, the one or more objects include one or more sport equipment comprising one or more of a ball, a club, a puck, and a hockey stick.

In some embodiments, the one or more biomechanical characteristics correspond to a wrist angle of the one or more users.

In some embodiments, the one or more physical activities correspond to a football sport. Further, the one or more physical activities include one or more of a quarterback throwing mechanic and a lineman blocking.

In some embodiments, the one or more physical activities correspond to a hockey sport. Further, the one or more physical activities include one or more of a shooting action and a goal-tending action.

In some embodiments, the retrieving of the one or more reference data includes retrieving two or more reference data based on the one or more content data. Further, the two or more reference data includes two or more reference metrics corresponding to the one or more physical activities. Further, the analyzing of the one or more characteristic data may be further based on the retrieving of the two or more reference data. Further, the analyzing of the one or more characteristic data includes analyzing the one or more characteristic data based on the two or more reference metrics.

In some embodiments, the generating of the feedback data may be further based on the one or more reference data and the one or more content data. Further, the generating of the feedback data includes generating an overlay content data based on the one or more reference data and the one or more content data. Further, the feedback data includes the overlay content data.

In some embodiments, the one or more reference data includes one or more reference content data comprising a reference video of the one or more physical activities. Further, the one or more content data includes an actual video of the one or more physical activities. Further, the overlay content data includes an overlay of the actual video on the reference video.

In some embodiments, the transmitting of the feedback data includes transmitting the feedback data to the one or more user devices 808 using a real-time communication protocol.

In some embodiments, the feedback data includes an actionable insight on the one or more physical activities.

In some embodiments, the feedback data includes the feedback on one or more of the two or more phases of the one or more physical activities.

In some embodiments, the one or more physical activities may be performed by one or more users. Further, the feedback on the one or more physical activities may be configured to facilitate improving a performance of the one or more users.

In some embodiments, the analyzing of the one or more content data includes analyzing the one or more biomechanical characteristics across the two or more phases of the one or more physical activities using the one or more AI models. Further, the determining of the one or more characteristic data may be based on the analyzing of the one or more biomechanical characteristics across the two or more phases of the one or more physical activities using the one or more AI models.

In some embodiments, the one or more AI models may be configured to track an incremental improvement on a performance of one or more users based on the analyzing of the one or more characteristic data. Further, the generating of the feedback data includes generating the feedback data using the one or more AI models based on the analyzing of the one or more characteristic data. Further, the feedback informs the improvement on the performance of the one or more users. Further, the one or more physical activities may be performed by the one or more users.

In some embodiments, the pocket phase includes preparation of a shot by aligning a ball with a shooting shoulder.

In some embodiments, the release phase occurs after the pocket phase. Further, the release phase includes releasing the ball from a hand. Further, the AI model may be configured to evaluate a wrist angle of one or more users during the release phase. Further, the one or more physical activities may be performed by the one or more users. Further, the one or more biomechanical characteristics include a wrist angle.

In some embodiments, the post-release phase occurs after the release phase. Further, the post-release phase includes a follow-through after the releasing of the ball from the hand. Further, the AI model may be configured to evaluate a body balance of the one or more users during the post-release phase. Further, the one or more biomechanical characteristics include the body balance of the one or more users.

In some embodiments, the feedback data indicates a progression of the one or more actual metrics towards the pre-injury phase. Further, the feedback on the one or more physical activities provides a positive reinforcement in relation to the progression of the one or more actual metrics.

In some embodiments, the one or more first feedback data includes one or more prior feedback data.

In some embodiments, the one or more AI models may be further configured to evaluate an interaction between two or more objects. Further, the one or more physical activities may be performed by the one or more users using the two or more objects.

In some embodiments, the one or more physical activities include a sport activity. Further, the two or more objects include one or more sport equipment comprising one or more of a ball, a club, a puck, and a hockey stick.

In some embodiments, the one or more characteristics include a trajectory of the ball, and an angle of a blade of the hockey stick during a slap shot.

In some embodiments, the one or more actual metrics of the one or more biomechanical characteristics indicate an alignment between a hand of the one or more users and the one or more objects.

In some embodiments, the one or more AI models may be configured to evaluate the two or more phases of the one or more physical activities using a rule-based method.

In some embodiments, the rule-based algorithm may be based on a heuristic profile comprising one or more of a confidence handling rule, a phase applicability flag, a personalization parameter, a threshold, and a weight.

FIG. 10 illustrates a flowchart 1000 of multi-phase evaluation of physical activities, in accordance with some embodiments. Further, the physical activities are performed by a player 1006 using one or more sports equipment. Further, the one or more sports equipment comprises one or more of a ball 1008 and a basket 1010. Further, a video of the physical activities of the player 1006 is captured by a camera 1004. Further, the multi-phase evaluation of the physical activities comprises segmentation 1012 of the video of the physical activities into two or more phases (1030-1034). Further, the two or more phases (1030-1034) comprise a phase one 1030, a phase two 1032, and a phase three 1034. Further, the multi-phase evaluation of the physical activities comprises scoring 1014 of each of the two or more phases (1030-1034). Further, the scoring 1014 is performed by an AI model to obtain two or more scores (1018-1022). Further, the AI model is configured to use a Gaussian distribution. Further, two or more scores (1018 1022) comprise a first score 1018 of the phase one 1030, a second score 1020 of the phase two 1032, and a third score 1022 of the phase three 1034. Further, the two or more scores (1018 1022) of the two or more phases (1030-1034) are presented on a user device 1016. Further, the two or more scores (1018-1022) of the two or more phases (1030-1034) are presented with two or more feedbacks (1024-1028) of the two or more phases (1030-1034). Further, the two or more feedbacks (1024-1028) comprise a first feedback 1024 corresponding to the phase one 1030, a second feedback 1026 corresponding to the phase two 1032, and a third feedback 1028 corresponding to the phase three 1034. Further, the two or more scores (1018-1022) and the two or more feedbacks (1024-1028) are presented on the user device 1016 with the video of the physical activities comprising the two or more phases (1030-1034).

FIG. 11 illustrates a spatial-temporal mechanics of a player 1100 playing Hockey, in accordance with some embodiments. Further, the spatial-temporal mechanics comprises one or more of a center of balance 1102, a transfer of weight 1104, an outside leg action 1106, a shoulder rotation 1108, a rotation of bottom axis 1110, a pull of top arm (flexion) 1112, a rotation of top hand 1114, a stick path 1116, a push position 1118, an ankle flexion inside leg 1120, a knee flexion inside leg 1122, a hip flexion inside leg 1124, a grip bottom hand 1126, a grip top hand 1128, a width of hands 1130, a puck impact point 1132, a tip control 1134, a shaft rotation 1136 (⅛-¼ turn), and a targeting-focus 1138.

FIG. 12 illustrates a graph 1200 of quaternion rotation dynamics, in accordance with some embodiments. Further, the graph 1200 depicts a change in the quaternion rotation dynamics associated with a basketball shot release. Further, the graph 1200 depicts the change in the quaternion rotation dynamics across two or more phases of the basketball shot release. Further, the two or more phases comprise an extension phase, a release phase, and a follow-through phase. Further, the quaternion rotation dynamics correspond to a wrist, a forearm, and an upper arm of a player performing the basketball shot release. Further, the quaternion rotation dynamics comprises one or more of a peak upper arm angular velocity, a maximum forearm extension rate, and a wrist snap peak velocity.

FIG. 13 illustrates a Gaussian distribution graph 1302 of physical activities, in accordance with some embodiments. Further, the Gaussian distribution graph 1302 comprises two or more Gaussian distribution graphs (1304-1308) corresponding to the two or more phases (1030 1034) of the physical activities. Further, the two or more Gaussian distribution graphs (1304 1308) comprise a first Gaussian distribution graph 1304 corresponding to the phase one 1030 of the physical activities, a second Gaussian distribution graph 1306 corresponding to the phase two 1032 of the physical activities, and a third Gaussian distribution graph 1308 corresponding to the phase three 1034 of the physical activities.

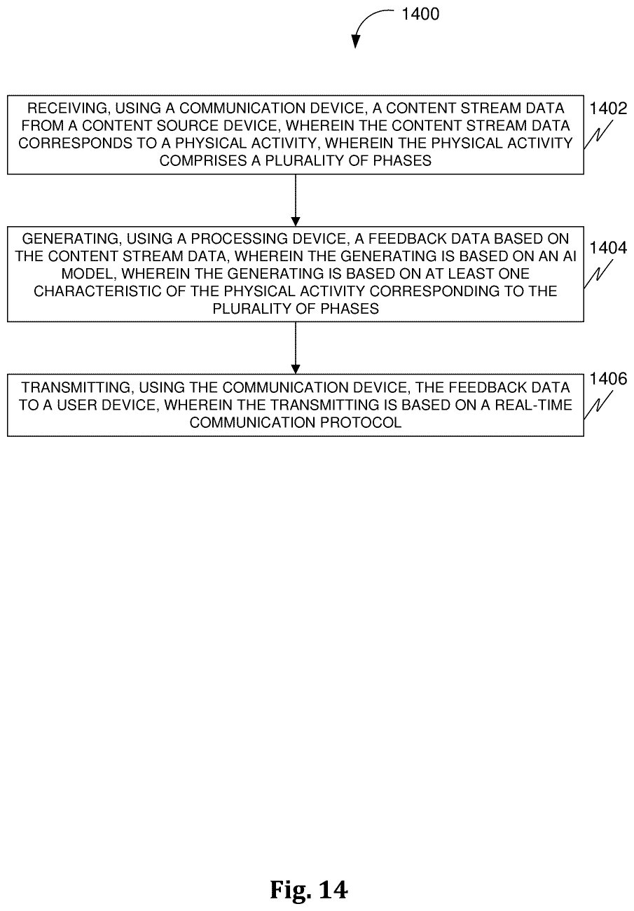

FIG. 14 illustrates a flowchart of a method 1400 of provisioning a performance evaluation of a physical activity, in accordance with some embodiments. Accordingly, the method 1400 may include a step 1402 of receiving, using a communication device 802, a content stream data from a content source device. Further, the content stream data corresponds to a physical activity. Further, the physical activity includes two or more phases. Further, the method 1400 may include a step 1404 of generating, using a processing device 804, a feedback data based on the content stream data. Further, the generating may be based on an AI model. Further, the generating may be based on two or more characteristics of the physical activity corresponding to the two or more phases. Further, the method 1400 may include a step 1406 of transmitting, using the communication device 802, the feedback data to a user device. Further, the transmitting may be based on a real-time communication protocol.

In some embodiments, the feedback data includes one or more metrics corresponding to the two or more characteristics. Further, the generating of the feedback data includes comparing the one or more metrics with one or more reference metrics. Further, the reference metric corresponds to the two or more characteristics.

In some embodiments, the two or more phases occur over two or more time intervals. Further, the two or more phases include a first phase and a second phase. Further, the two or more time intervals include a first time interval corresponding to the first phase and a second time interval corresponding to the second phase. Further, the second time interval occurs later than the first time interval.

In some embodiments, the physical activity corresponds to a sport. Further, the physical activity may be associated with two or more objects. Further, the two or more objects includes a player performing the physical activity and a sport object. Further, the plurality characteristic includes a player characteristic and a sport object characteristic. Further, the player characteristic corresponds to the player. Further, the sport object characteristic corresponds to the sport object. Further, the sport object characteristic may be based on the player characteristic.

In some embodiments, the method 1400 may further include storing, using a storage device 902, the feedback data. Further, the physical activity includes a first physical activity corresponding to a first time period and a second physical activity corresponding to a second time period. Further, the second time period occurs later than the first time period. Further, the feedback data includes a first feedback data corresponding to the first time period and a second feedback data corresponding to the second time period. Further, the generating of the second feedback data may be based further on the first feedback data.

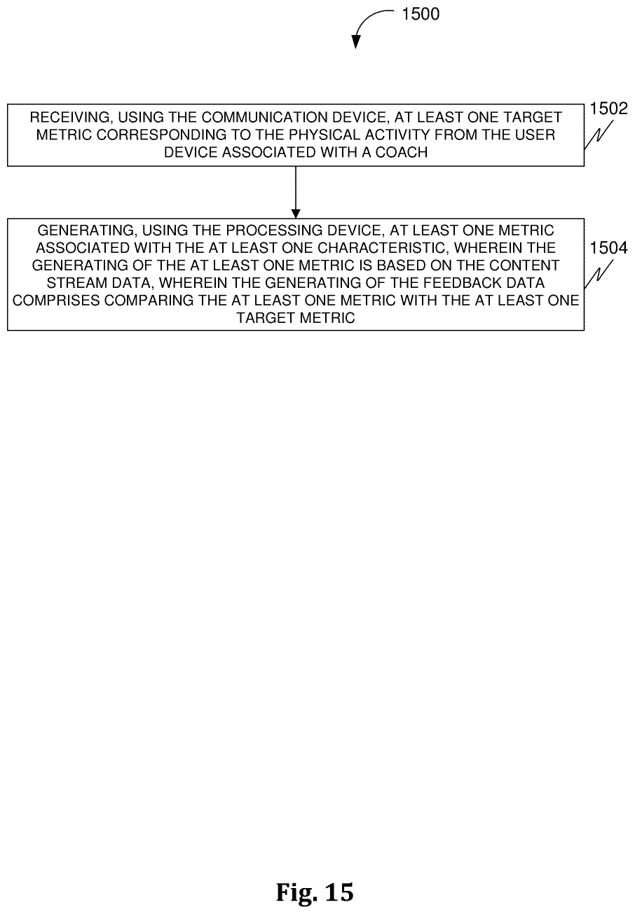

FIG. 15 illustrates a flowchart of a method 1500 of provisioning a performance evaluation of a physical activity including generating, using the processing device 804, at least one metric associated with the plurality of characteristics, in accordance with some embodiments. Further, in some embodiments, the method 1500 further may include a step 1502 of receiving, using the communication device 802, one or more target metric corresponding to the physical activity from the user device associated with a coach. Further, in some embodiments, the method 1500 further may include a step 1504 of generating, using the processing device 804, one or more metrics associated with the two or more characteristics. Further, the generating of the one or more metrics may be based on the content stream data. Further, the generating of the feedback data includes comparing the one or more metrics with the one or more target metrics.

In some embodiments, the first physical activity corresponds to a pre-injury state. Further, the second physical activity corresponds to an injury recovery state. Further, the first feedback data corresponds to the pre-injury state. Further, the second feedback data corresponds to the injury recovery state.

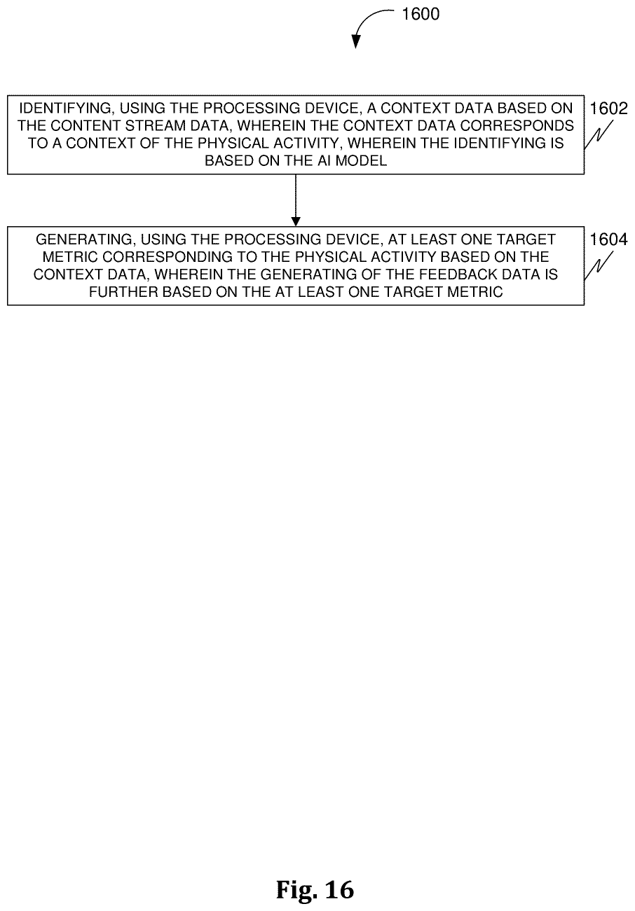

FIG. 16 illustrates a flowchart of a method 1600 of provisioning a performance evaluation of a physical activity including generating, using the processing device 804, at least one target metric corresponding to the physical activity, in accordance with some embodiments. Further, in some embodiments, the method 1600 further may include a step 1602 of identifying, using the processing device 804, a context data based on the content stream data. Further, the context data corresponds to a context of the physical activity. Further, the identifying may be based on the AI model. Further, in some embodiments, the method 1600 further may include a step 1604 of generating, using the processing device 804, one or more target metrics corresponding to the physical activity based on the context data. Further, the generating of the feedback data may be further based on the one or more target metrics.

In some embodiments, the method 1400 may further include analyzing, using the processing device 804, the content stream data based on a three-dimensional spatial data corresponding to three-dimensional modeling of one or more of the physical activity and a player performing the physical activity. Further, the generating of feedback data may be further based on the analyzing.

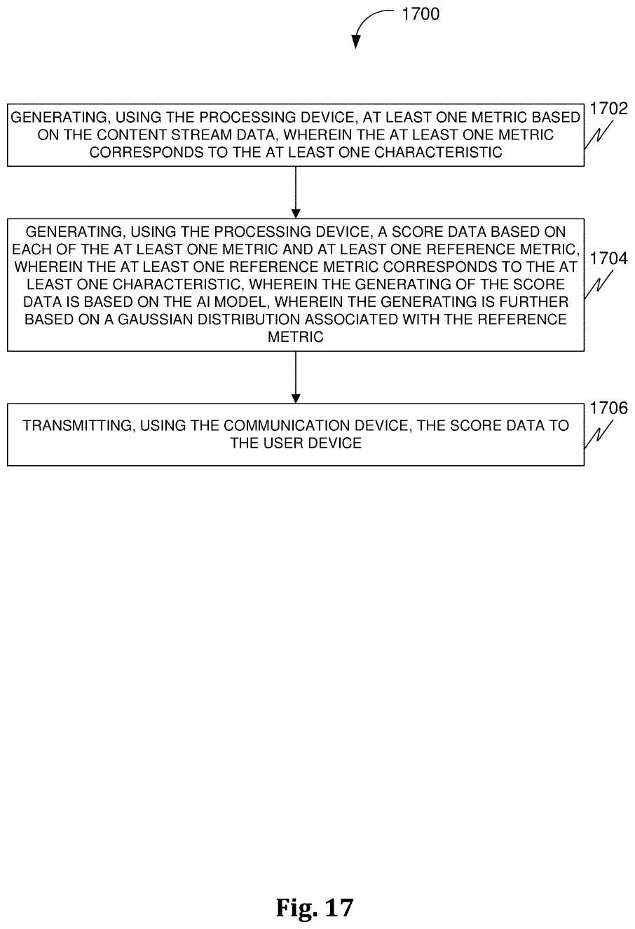

FIG. 17 illustrates a flowchart of a method 1700 of provisioning a performance evaluation of a physical activity including generating, using the processing device 804, a score data, in accordance with some embodiments. Further, in some embodiments, the method 1700 further may include a step 1702 of generating, using the processing device 804, one or more metrics based on the content stream data. Further, the one or more metrics corresponds to the two or more characteristics. Further, in some embodiments, the method 1700 further may include a step 1704 of generating, using the processing device 804, a score data based on each of the one or more metrics and one or more reference metrics. Further, the one or more reference metrics correspond to the two or more characteristics. Further, the generating of the score data may be based on the AI model. Further, the generating may be further based on a Gaussian distribution associated with the reference metric. Further, in some embodiments, the method 1700 further may include a step 1706 of transmitting, using the communication device 802, the score data to the user device.

Accordingly, the system 800 may include a communication device 802. Further, the communication device 802 may be configured for receiving a content stream data from a content source device. Further, the content stream data corresponds to a physical activity. Further, the physical activity includes two or more phases. Further, the communication device 802 may be configured for transmitting a feedback data to a user device. Further, the transmitting may be based on a real-time communication protocol. Further, the system 800 may include a processing device 804 which may be configured for generating the feedback data based on the content stream data. Further, the generating may be based on an AI model. Further, the generating may be based on two or more characteristics of the physical activity corresponding to the two or more phases.

In some embodiments, the feedback data includes one or more metrics corresponding to the two or more characteristics. Further, the generating of the feedback data includes comparing the one or more metrics with one or more reference metrics. Further, the reference metric corresponds to the two or more characteristics.

In some embodiments, the two or more phases occur over two or more time intervals. Further, the two or more phases include a first phase and a second phase. Further, the two or more time intervals include a first time interval corresponding to the first phase and a second time interval corresponding to the second phase. Further, the second time interval occurs later than the first time interval.

In some embodiments, the physical activity corresponds to a sport. Further, the physical activity may be associated with two or more objects. Further, the two or more objects include a player performing the physical activity and a sport object. Further, the plurality characteristic includes a player characteristic and a sport object characteristic. Further, the player characteristic corresponds to the player. Further, the sport object characteristic corresponds to the sport object. Further, the sport object characteristic may be based on the player characteristic.

In some embodiments, the system 800 may further include a storage device 902 which may be configured for storing the feedback data. Further, the physical activity includes a first physical activity corresponding to a first time period and a second physical activity corresponding to a second time period. Further, the second time period occurs later than the first time period. Further, the feedback data includes a first feedback data corresponding to the first time period and a second feedback data corresponding to the second time period. Further, the generating of the second feedback data may be based further on the first feedback data.

In some embodiments, the communication device 802 may be further configured for receiving one or more target metrics corresponding to the physical activity from the user device associated with a coach. Further, the processing device 804 may be further configured for generating one or more metrics associated with the two or more characteristics. Further, the generating of the one or more metrics may be based on the content stream data. Further, the generating of the feedback data includes comparing the one or more metrics with the one or more target metrics.

In some embodiments, the first physical activity corresponds to a pre-injury state. Further, the second physical activity corresponds to an injury recovery state. Further, the first feedback data corresponds to the pre-injury state. Further, the second feedback data corresponds to the injury recovery state.

Further, in some embodiments, the processing device 804 may be further configured for identifying a context data based on the content stream data. Further, the context data corresponds to a context of the physical activity. Further, the identifying may be based on the AI model. Further, the processing device 804 may be further configured for generating one or more target metric corresponding to the physical activity based on the context data. Further, the generating of the feedback data may be further based on the one or more target metrics.

In some embodiments, the processing device 804 may be further configured for analyzing the content stream data based on a three-dimensional spatial data corresponding to three-dimensional modeling of one or more of the physical activity and a player performing the physical activity. Further, the generating of feedback data may be further based on the analyzing.

Further, in some embodiments, the processing device 804 may be further configured for generating one or more metrics based on the content stream data. Further, the one or more metrics corresponds to the two or more characteristics. Further, the processing device 804 may be further configured for generating a score data based on each of the one or more metrics and one or more reference metrics. Further, the one or more reference metrics correspond to the two or more characteristics. Further, the generating of the score data may be based on the AI model. Further, the generating may be further based on a Gaussian distribution associated with the reference metric. Further, the communication device 802 may be further configured for transmitting the score data to the user device.

In some embodiments, the content stream data includes two or more content stream data. Further, the two or more content stream data corresponds to the two or more phases.

In some embodiments, the content stream data corresponds to a performance of the physical activity.

In some embodiments, the physical activity includes a sports activity.

In some embodiments, the physical activity may be performed by a user. Further, the user corresponds to a sport player. In some embodiments, the physical activity corresponds to Basketball. Further, the two or more phases include one or more of a pre-release phase, a release phase, and a post-release phase.

In some embodiments, the physical activity corresponds to Football. Further, the physical activity includes one or more of a quarterback throwing activity and a phases of the quarterback throwing activity. Further, the physical activity includes one or more of a lineman blocking activity and a phases of the lineman blocking activity.

In some embodiments, the physical activity corresponds to Hockey. Further, the physical activity includes one or more of a shooting activity and a phases of the shooting activity. Further, the physical activity includes one or more of a goal-tending activity and a phases of the goal-tending activity.

In some embodiments, the first feedback data includes one or more reference metrics corresponding to a historical performance. Further, the second feedback data represents an improvement in the physical activity over time.

In some embodiments, the one or more target metrics correspond to a sport coaching target.

In some embodiments, the second feedback data represents one or more of an increment and a decrement of the performance over time.

In some embodiments, the receiving may be based on the real-time communication protocol.

In some embodiments, the real-time communication protocol corresponds to one or more of a WebRTC (or similar), a Websockets (or similar), a real-time streaming protocol, a real-time transfer protocol and a live-streaming protocol.

In some embodiments, the two or more characteristics may be based on the context.

In some embodiments, the feedback data includes the one or more metrics associated with the two or more characteristics. Further, the generating of the feedback data includes comparing the one or more metrics with the one or more target metrics.

In some embodiments, the content stream data includes Basketball. Further, the context corresponds to each of a guarded shot and an unguarded shot. Further, the two or more characteristics include a first characteristic associated with the guarded shot and a second characteristic associated with the unguarded shot. Further, the generating of the feedback data includes comparing a first characteristic data with the second characteristic data. Further, the first characteristic data represents the first characteristic. Further, the second characteristic data represents the second characteristic.

In some embodiments, the feedback data includes two or more feedback data. Further, the two or more feedback data may be based on two or more reference metrics. Further, the two or more reference metrics correspond to the two or more characteristics.

In some embodiments, the user device may be configured to present the two or more feedback data concurrently.

In some embodiments, the three-dimensional spatial data correspond to a three dimensional spatial orientation, arrangement, separation etc.

In some embodiments, the analyzing further comprising generating one or more metrics corresponding to the two or more characteristics. Further, the two or more characteristics include a biomechanical variable associated with the physical activity.

In some embodiments, the biomechanical variable corresponds to a joint angle of a player performing the physical activity. Further, the joint angle corresponds to angle formed between two body parts of the player. Further, the two body parts may be linked by a joint.

In some embodiments, the biomechanical variable corresponds to a body alignment of a player performing the physical activity. Further, the body alignment corresponds to positioning of body parts of the player.

In some embodiments, the player characteristic and a sport object characteristic corresponds to a movement trajectory of the player performing the physical activity and a movement trajectory of the sport object respectively. Further, the movement trajectory may be a representation of a path that the player and the sport object follows over a time.

In some embodiments, the generating further comprising determining a deviation of the one or more metrics from the one or more reference metrics based on the Gaussian distribution.

In some embodiments, the player characteristic includes a biomechanical characteristic. Further, the biomechanical characteristic corresponds to a mechanics of a player.

In some embodiments, the sport object characteristic includes a mechanics of a sport object.

In some embodiments, the sport object corresponds to a ball.

In some embodiments, the sport object corresponds to a hockey stick.

In some embodiments, the sport object corresponds to a golf club. Further, the golf club may be used to hit a ball in a Golf sport.

In some embodiments, the sport object corresponds to a baseball bat.

In some embodiments, the three dimensional spatial data includes a quaternion. Further, the quaternion includes one or more of a vector representation and a scalar representation.

In some embodiments, the two or more characteristic corresponds to a rotational orientation of a player performing the physical activity. Further, the rotational orientation may be represented using the quaternions. Further, the vector representation represents an axis of rotation. Further, in the scalar representation represents one or more of a magnitude and an angle of rotation.

In some embodiments, the quaternions encode the three-dimensional orientation of body parts or joints of the player, providing a singularity-free representations of rotational data.

In some embodiments, the two or more characteristics correspond to a relative orientation between the two body parts of a player. Further, the two body parts may be linked by a joint. Further, the relative orientation may be represented as a quaternion rotation.

In some embodiments, the determining of a joint angle, angular velocities and a deviation from the reference metric may be further based on the relative orientation.

In some embodiments, the generating of the one or more metrics based on one or more quaternion calculations. Further, the quaternion calculation may be based on one or more of an angular velocity, an orientation difference, and a rotation calculation, the one or more metrics may be used to evaluate one or more of a body alignment of a player performing the physical activity and a movement of the player performing the physical activity.

In some embodiments, the content stream data corresponds to a Hockey. Further, the sport object characteristic corresponds to an angle of a hockey stick.

In some embodiments, the metric data includes two or more metric data. Further, the metric reference data includes two or more metric reference data. Further, each of the two or more metric data and the two or more metric reference data corresponds to the two or more phases.

In some embodiments, the generating of the feedback data may be further based on the score data.

In some embodiments, the feedback data represents one or more of an improvement, a decrement and a deviation in a performance of the physical activity over time.

In some embodiments, the feedback data represents a progress in a performance of the physical activity over time.

In some embodiment, the feedback data represents a statement that encourages a user. In some embodiments, the physical activity may correspond to Basketball shot. Further, the one or more characteristic may correspond to a wrist mechanics of a player performing the physical activity. Further, each of the one or more reference metric data and the one or more metric data corresponds to a wrist angle.

In some embodiments, the physical activity may correspond to Hockey. Further, the one or more characteristic corresponds to a body alignment of a player performing the physical activity.

In some embodiments, the physical activity includes Football blocking action. Further, the two or more characteristics correspond to a body balance and the body posture of a player performing the physical activity.

In some embodiments, the physical activity includes Hockey save action. Further, the one or more characteristic correspond to a body balance and the body posture of a player performing the physical activity.

In some embodiments, the user device may be configured to present the feedback data over a video data.

In some embodiments, the content stream data corresponds to a video data.

In some embodiments, the content source device is and/or includes a camera.

In some embodiments, the method 1400 may further include retrieving, using a storage device 902, the one or more reference metric data based on the content stream data.

In some embodiments, the generating of the feedback data may be based on a rule-based algorithm.

In some embodiments, the rule-based algorithm corresponds to a heuristic algorithm.

In some embodiments, the heuristic algorithm may be based on certain rules to generate the feedback data.

In some embodiments, the feedback data represents a consistency in performing the physical activity over time.

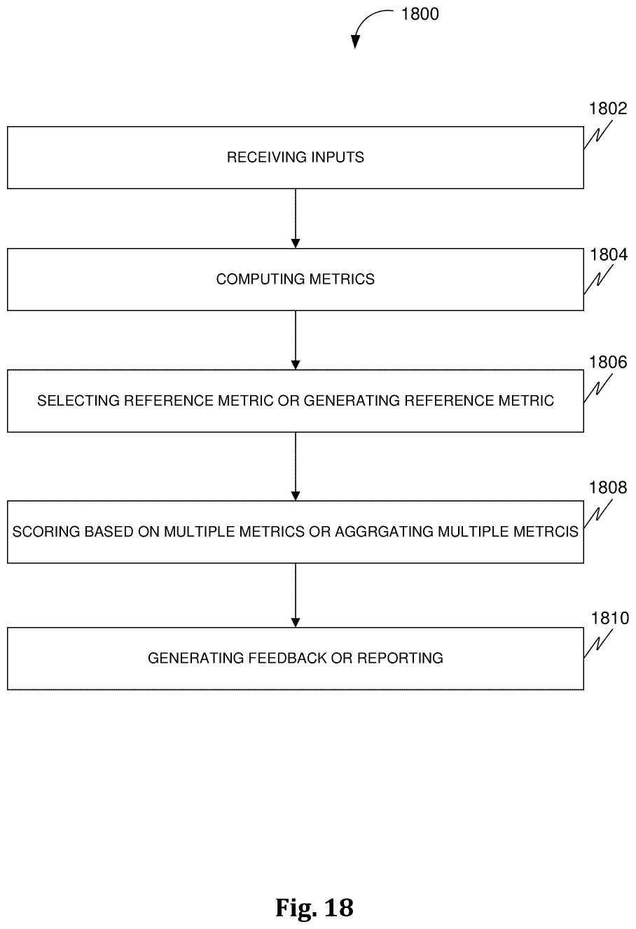

FIG. 18 illustrates a flowchart of a method 1800 of facilitating performance evaluation of physical activities, in accordance with some embodiments. Further, the method 1800 may include a step 1802 of receiving inputs. Further, the inputs comprise one or more of a pose data, a quaternion data, and an object data. Further, the method 1800 may include a step 1804 of computing metrics of biomechanical characteristics based on the inputs. Further, the method 1800 may include a step 1806 of one or more of selecting a reference metric and generating the reference metric. Further, the method 1800 may include a step 1808 of performing one or more of scoring based on two or more metrics and aggregating the two or more metrics. Further, the method 1800 may include a step 1810 of one or more of generating feedback and reporting.

Although the invention has been explained in relation to its preferred embodiment, it is to be understood that many other possible modifications and variations can be made without departing from the spirit and scope of the invention as hereinafter claimed.

What is claimed is:

1. A method of facilitating performance evaluation of physical activities, the method comprising:
receiving, using a communication device, at least one content data from at least one content source device, wherein the at least one content data corresponds to at least one physical activity;
determining, using a processing device, at least one characteristic data using at least one Artificial Intelligence (AI) model based on the at least one content data, wherein the at least one characteristic data comprises at least one actual metric of at least one biomechanical characteristic of the at least one physical activity;
analyzing, using the processing device, the at least one characteristic data and at least one reference data, wherein the at least one reference data comprises at least one reference metric of the at least one biomechanical characteristic of the at least one physical activity;
generating, using the processing device, a feedback data based on the analyzing of the at least one characteristic data, wherein the feedback data comprises a feedback on the at least one physical activity; and
transmitting, using the communication device, the feedback data to at least one user device.

2. The method of claim 1, wherein the at least one physical activity comprises a plurality of phases, wherein the at least one AI model is configured to evaluate the at least one biomechanical characteristic of the at least one physical activity comprising the plurality of phases, wherein the method further comprises analyzing, using the processing device, the at least one content data using the at least one AI model, wherein the determining of the at least one characteristic data is further based on the analyzing of the at least one content data using the at least one AI model.

3. The method of claim 1 further comprising determining, using the processing device, a deviation of the at least one actual metric from the at least one reference metric based on the analyzing of the at least one characteristic data and the at least one reference data, wherein the generating of the feedback data is further based on the determining of the deviation.

4. The method of claim 3, wherein the generating of the feedback data comprises generating at least one score using the at least one AI model based on the determining of the deviation, wherein the at least one AI model is configured to generate the at least one score for the deviation based on a Gaussian distribution associated with the at least one reference metric, wherein the feedback data comprises the at least one score.

5. The method of claim 1 further comprising retrieving, using a storage device, the at least one reference data from a database based on the at least one content data, wherein the database comprises a plurality of reference data corresponding to a plurality of physical activities, wherein the analyzing of the at least one characteristic data is further based on the retrieving of the at least one reference data.

6. The method of claim 1, wherein the at least one reference data comprises at least one first feedback data corresponding to at least one first content data, wherein the at least one first content data corresponds to at least one first physical activity, wherein the at least one first content data is received at a first time instance, wherein the at least one content data is received at a second time instance, wherein the second time instance occurs after the first time instance, wherein the at least one reference metric comprises at least one first actual metric corresponding to the at least one biomechanical characteristic of the at least one first physical activity.

7. The method of claim 1 further comprising:
receiving, using the communication device, at least one target data from the at least one user device, wherein the at least one target data comprises at least one target information corresponding to the at least one physical activity;
retrieving, using a storage device, at least one first feedback data based on the at least one content data, wherein the at least one first feedback data corresponds to at least one first content data corresponding to at least one first physical activity, wherein the at least one first feedback data comprises at least one first actual metric of the at least one biomechanical characteristic of the at least one first physical activity, wherein the at least one first content data is received at a first time instance, wherein the at least one content data is received at a second time instance, wherein the second time instance occurs after the first time instance; and
generating, using the processing device, the at least one reference data based on the at least one first feedback data and the at least one target data, wherein the at least one reference metric is based on each of the at least one first actual metric and the at least one target information, wherein the analyzing of the at least one characteristic data is further based on the generating of the at least one reference data.

8. The method of claim 5 further comprising:
receiving, using the communication device, at least one scenario data from the at least one user device, wherein the at least one scenario data indicates at least one scenario of the at least one physical activity; and
identifying, using the processing device, the at least one reference data from the plurality of reference data based on the at least one scenario data, wherein the at least one reference data is associated with the at least one scenario data, wherein the retrieving of the at least one reference data is based on the identifying of the at least one reference data, wherein the plurality of reference data corresponds to the plurality of physical activities associated with a plurality of scenarios.

9. The method of claim 1 further comprising:
receiving, using the communication device, an injury phase data from the at least one user device, wherein the injury phase data indicates at least one injury phase of at least one user performing the at least one physical activity; and
generating, using the processing device, the at least one reference data based on the injury phase data, wherein the at least one reference data comprises the at least one reference metric of the at least one biomechanical characteristic associated with the at least one injury phase, wherein the analyzing of the at least one characteristic data is further based on the generating of the at least one reference data.

10. The method of claim 1 further comprising generating, using the processing device, at least one spatial data based on the at least one content data, wherein the at least one spatial data comprises at least one three-dimensional metric corresponding to the at least one physical activity, wherein the determining of the at least one characteristic data is further based on the at least one spatial data, wherein the at least one AI model is configured to determine the at least one biomechanical characteristic of the at least one physical activity based on the at least one spatial data.

11. A system for facilitating performance evaluation of physical activities, the system comprising:
a communication device configured for:
receiving at least one content data from at least one content source device, wherein the at least one content data corresponds to at least one physical activity; and
transmitting a feedback data to at least one user device; and
a processing device communicatively coupled with the communication device, wherein the processing device is configured for:
determining at least one characteristic data using at least one Artificial Intelligence (AI) model based on the at least one content data, wherein the at least one characteristic data comprises at least one actual metric of at least one biomechanical characteristic of the at least one physical activity;
analyzing the at least one characteristic data and at least one reference data, wherein the at least one reference data comprises at least one reference metric of the at least one biomechanical characteristic of the at least one physical activity; and
generating the feedback data based on the analyzing of the at least one characteristic data, wherein the feedback data comprises a feedback on the at least one physical activity.

12. The system of claim 11, wherein the at least one physical activity comprises a plurality of phases, wherein the at least one AI model is configured to evaluate the at least one biomechanical characteristic of the at least one physical activity comprising the plurality of phases, wherein the processing device is further configured for analyzing the at least one content data using the at least one AI model, wherein the determining of the at least one characteristic data is further based on the analyzing of the at least one content data using the at least one AI model.

13. The system of claim 11, wherein the processing device is further configured for determining a deviation of the at least one actual metric from the at least one reference metric based on the analyzing of the at least one characteristic data and the at least one reference data, wherein the generating of the feedback data is further based on the determining of the deviation.

14. The system of claim 13, wherein the generating of the feedback data comprises generating at least one score using the at least one AI model based on the determining of the deviation, wherein the at least one AI model is configured to generate the at least one score for the deviation based on a Gaussian distribution associated with the at least one reference metric, wherein the feedback data comprises the at least one score.

15. The system of claim 11 further comprising a storage device communicatively coupled with the communication device, wherein the storage device is configured for retrieving the at least one reference data from a database based on the at least one content data, wherein the database comprises a plurality of reference data corresponding to a plurality of physical activities, wherein the analyzing of the at least one characteristic data is further based on the retrieving of the at least one reference data.

16. The system of claim 11, wherein the at least one reference data comprises at least one first feedback data corresponding to at least one first content data, wherein the at least one first content data corresponds to at least one first physical activity, wherein the at least one first content data is received at a first time instance, wherein the at least one content data is received at a second time instance, wherein the second time instance occurs after the first time instance, wherein the at least one reference metric comprises at least one first actual metric corresponding to the at least one biomechanical characteristic of the at least one first physical activity.

17. The system of claim 11, wherein the communication device is further configured for receiving at least one target data from the at least one user device, wherein the at least one target data comprises at least one target information corresponding to the at least one physical activity, wherein the system further comprises a storage device communicatively coupled with the communication device, wherein the storage device is configured for retrieving at least one first feedback data based on the at least one content data, wherein the at least one first feedback data corresponds to at least one first content data of the at least one first physical activity, wherein the at least one first feedback data comprises at least one first actual metric corresponding to the at least one biomechanical characteristic of the at least one first physical activity, wherein the at least one first content data is received at a first time instance, wherein the at least one content data is received at a second time instance, wherein the second time instance occurs after the first time instance, wherein the processing device is further communicatively coupled with the storage device, wherein the processing device is configured for generating the at least one reference data based on the at least one first feedback data and the at least one target data, wherein the at least one reference metric is based on each of the at least one first actual metric and the at least one target information, wherein the analyzing of the at least one characteristic data is further based on the generating of the at least one reference data.

18. The system of claim 15, wherein the communication device is further configured for receiving at least one scenario data from the at least one user device, wherein the at least one scenario data indicates at least one scenario of the at least one physical activity, wherein the processing device is configured for identifying the at least one reference data form the plurality of reference data based on the at least one scenario data, wherein the at least one reference data is associated with the at least one scenario data, wherein the retrieving of the at least one reference data is based on the identifying of the at least one reference data, wherein the plurality of reference data corresponds to the plurality of physical activities associated with a plurality of scenarios.

19. The system of claim 11, wherein the communication device is further configured for receiving an injury phase data from the at least one user device, wherein the injury phase data indicates at least one injury phase of at least one user performing the at least one physical activity, wherein the processing device is further configured for generating the at least one reference data based on the injury phase data, wherein the at least one reference data comprises the at least one reference metric of the at least one biomechanical characteristic associated with the at least one injury phase, wherein the analyzing of the at least one characteristic data is further based on the generating of the at least one reference data.

20. The system of claim 11, wherein the processing device is further configured for generating at least one spatial data based on the at least one content data, wherein the at least one spatial data comprises at least one three-dimensional metric corresponding to the at least one physical activity, wherein the determining of the at least one characteristic data is further based on the at least one spatial data, wherein the at least one AI model is configured to determine the at least one biomechanical characteristic of the at least one physical activity based on the at least one spatial data.
